(19) United States
(12) Patent Application Publication
(10) Pub. No.: US 2026/0272421 A1
(43) Pub. Date: Sep. 17, 2026

(54) METHODS, SYSTEMS, AND APPARATUSES FOR MEASURING DEPTH-RESOLVED TISSUE KINEMATICS

(71) Applicant: George Mason University, Fairfax, VA (US)

(72) Inventors: Siddhartha Sikdar, Washington, DC (US); Parag Vijay Chitnis, Burke, VA (US); Ahmed Bashatah, Fairfax, VA (US); Abhishek Sanjay Aher, Fairfax, VA (US)

(21) Appl. No.: 19/447,757

(22) Filed: Jan. 13, 2026

Related U.S. Application Data
(60) Provisional application No. 63/771,201, filed on Mar. 13, 2025.

(51) Int. Cl. A61B 8/00; G01S 15/89

(52) U.S. Cl. CPC A61B 8/488; A61B 8/4236; A61B 8/486; G01S 15/8977

(57) ABSTRACT

Methods, systems, and apparatuses for employing frequency-modulated continuous wave signals to simultaneously measure tissue depth and velocity using a single transducer element are described. The system operates at low voltages and utilizes time-delay spectrometry techniques to extract both range and kinematic information from backscattered ultrasound signals. This approach enables real-time monitoring of muscle movement and blood flow during dynamic activities without requiring precise transducer placement or complex image processing. The compact, energy-efficient design makes the system suitable for applications in rehabilitation monitoring, athletic performance assessment, and control of powered prosthetic and orthotic devices, among other applications.

### CROSS REFERENCE TO RELATED PATENT APPLICATION

This application claims priority to U.S. Provisional Application No. 63/771,201, filed Mar. 13, 2025, which is herein incorporated by reference in its entirety.

### FEDERALLY SPONSORED RESEARCH AND DEVELOPMENT

This invention was made with government support under grant number EB027601 awarded by the National Institutes of Health, and grant numbers W81XWH-22-9-0016 and W81XWH-20-1-0817 awarded by the DHA/MRDB. The government has certain rights in the invention.

### BACKGROUND

Ultrasound imaging has become a widely adopted medical imaging modality due to its non-ionizing radiation properties, portability, and relatively low cost compared to other imaging techniques. Conventional ultrasound systems operate in various modes including A-mode for single scan line visualization, M-mode for tissue movement tracking along a single line over time, B-mode for real-time anatomical structure imaging, and Doppler modes for detecting and quantifying moving blood and tissue.

Recent developments in ultrasound technology have expanded applications beyond conventional clinical imaging to include blood pressure monitoring, muscle activity tracking during dynamic tasks, and control signal generation for powered prosthetic and orthotic devices. These applications often require continuous monitoring of tissue motion and muscle activation patterns during physical activities, rehabilitation exercises, and athletic performance assessment.

Current wearable ultrasound systems face several technical limitations that restrict their practical implementation. These systems typically rely on pulse-echo imaging techniques that require high-voltage transmission signals ranging from 20 to 100 volts peak-to-peak and short-duration pulses in the nanosecond to microsecond range. Such high-voltage requirements necessitate specialized front-end electronics, transmit/receive switches, and high-speed digitization systems operating at 20 to 100 mega-samples per second. These hardware demands create substantial constraints on miniaturization and power consumption, making truly wearable implementations challenging. High voltage transmission signals may raise safety concerns for applications with prolonged skin contact.

Conventional Doppler ultrasound systems present additional technical challenges for wearable applications. Continuous wave Doppler systems require separate transmit and receive transducers positioned at predetermined angles, complicating the development of compact multi-channel wearable devices. Pulsed wave Doppler systems, while providing depth discrimination, require high-speed digitizers for precise phase tracking. Furthermore, these systems lack the ability to measure depth-resolved tissue kinematics using a single transducer element operating at a low voltage. Tissue kinematics refers to a number of variables describing tissue motion: tissue speed and its time integral (tissue displacement); first and second order time derivatives of speed (acceleration and jerk, respectively); and spatial derivatives of speed (strain rate) and displacement (strain). Both the magnitude and direction of tissue kinematic variables are of interest in many applications, including but not limited to biomechanics, sports science, rehabilitation, and other disciplines involved in the diagnosis, treatment and monitoring of neuromusculoskeletal disorders, and functional assessment and improvement.

Emerging wearable ultrasound imaging systems often require precise transducer positioning by trained personnel to generate useful data, and complex image processing algorithms are needed to extract meaningful metrics from the ultrasound signals. These challenges of placement sensitivity and operator skill requirements need to be overcome to enable ultrasound-based monitoring to be used beyond specialized clinical environments and trained operators.

Existing approaches to tissue motion monitoring during dynamic physical activities suffer from limitations in providing real-time, simultaneous measurement of both tissue depth and its movement kinematics. The inability to obtain both range and kinematic information from a single, low-voltage transducer element represents a technical gap that hinders the development of practical wearable ultrasound systems for applications including but not limited to physiological monitoring (e.g., for cardiovascular and rehabilitation applications), functional assessment (e.g., for assessing athletic performance or fitness of military personnel), and human-machine interfaces (e.g., using these signals for inferring motor intent for control of powered prosthetic and orthotic devices or for gesture recognition).

### SUMMARY

It is to be understood that both the following general description and the following detailed description are exemplary and explanatory only and are not restrictive.

Methods, apparatuses, and systems for employing frequency-modulated continuous wave signals to simultaneously measure depth-resolved tissue kinematics using a single transducer element are described. The system operates at low voltages and utilizes time-delay spectrometry techniques to extract both range and Doppler information from backscattered ultrasound signals. This approach enables real-time monitoring of tissue movement and blood flow during dynamic activities without requiring precise transducer placement or complex image processing. The compact, energy-efficient design makes the system suitable for applications in rehabilitation monitoring, athletic performance and military fitness assessment, and control of powered prosthetic and orthotic devices, among other applications.

In an embodiment, disclosed are methods comprising receiving, via one or more single transducers positioned on one or more locations of an individual, based on a transmission of one or more ultrasound signals, one or more backscattered ultrasound signals, generating, based on the one or more ultrasound signals and the one or more backscattered ultrasound signals, a mixed signal, generating, based on applying a lowpass filter to the mixed signal, an intermediate frequency signal, and extracting, based on processing the intermediate frequency signal, range information associated with tissue depth associated with the one or more locations and kinematic information associated with range-resolved tissue velocity associated with the one or more locations.

In an embodiment, disclosed are apparatuses comprising one or more processors, and a memory storing processor-executable instructions that, when executed by the one or more processors, cause the apparatus to receive, via one or more single transducers positioned on one or more locations of an individual, based on a transmission of one or more ultrasound signals, one or more backscattered ultrasound signals, generate, based on the one or more ultrasound signals and the one or more backscattered ultrasound signals, a mixed signal, generate, based on applying a lowpass filter to the mixed signal, an intermediate frequency signal, and extract, based on processing the intermediate frequency signal, range information associated with tissue depth associated with the one or more locations and kinematic information associated with range-resolved tissue velocity associated with the one or more locations.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system for measuring tissue depth and speed;

FIG. 2 shows an example ultrasound signal with frequency modulated characteristics;

FIG. 3A-3B show example ultrasound signals with linear chirp characteristics;

FIGS. 4A-4B show example spectra of intermediate frequency signals without and with tissue motion;

FIGS. 5A-5D show examples of data associated with a process of analyzing the frequency domain;

FIGS. 6A-6C show examples of frequency domain analysis of an intermediate frequency signal obtained from moving tissue to yield power spectral density data;

FIGS. 7A-7D shows examples of a time-domain analysis of an intermediate frequency signal obtained from moving tissue;

FIG. 8 shows an example experimental comparison between analyzed Doppler frequency shifts different demodulation methods;

FIG. 9 shows an example system environment for measuring tissue depth and kinematics;

FIG. 10 shows example system architecture;

FIG. 11 shows example system architecture;

FIG. 12 shows an example process;

FIGS. 13A-13D show example processes;

FIG. 14 shows example system architecture;

FIG. 15 shows example system architecture;

FIGS. 16A-16L show example muscle velocity data;

FIG. 17 shows example movement velocity vector data;

FIGS. 18A-18E show example muscle displacement and velocity data;

FIGS. 19A-19E show example muscle displacement and velocity data;

FIG. 20A shows example blood velocity data;

FIG. 20B shows example tissue velocity and displacement data;

FIG. 21 shows an example prosthetic that may be controlled based on information output by one or more transducers;

FIGS. 22A-22T show example Doppler images associated with a residual forearm of an individual; and

FIG. 23 shows a flowchart of an example process for measuring tissue depth and velocities.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

As used herein the terms “individual,” “patient,” “subject,” “user,” or “person” may indicate a person associated with the determination of one or more physiological parameters, such as an interstitial insulin elimination rate, a hepatic insulin elimination rate, a renal insulin elimination rate, or a permeability constant related to insulin diffusion in the human body.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, the methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks. In addition, some of these functions may be carried out using complex programmable logic devices (CPLDs) or other programmable logic devices.

The processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks. In addition, some of these functions may be carried out using logic devices which do not operate by sequential operations of programmed steps.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions and logic circuitry.

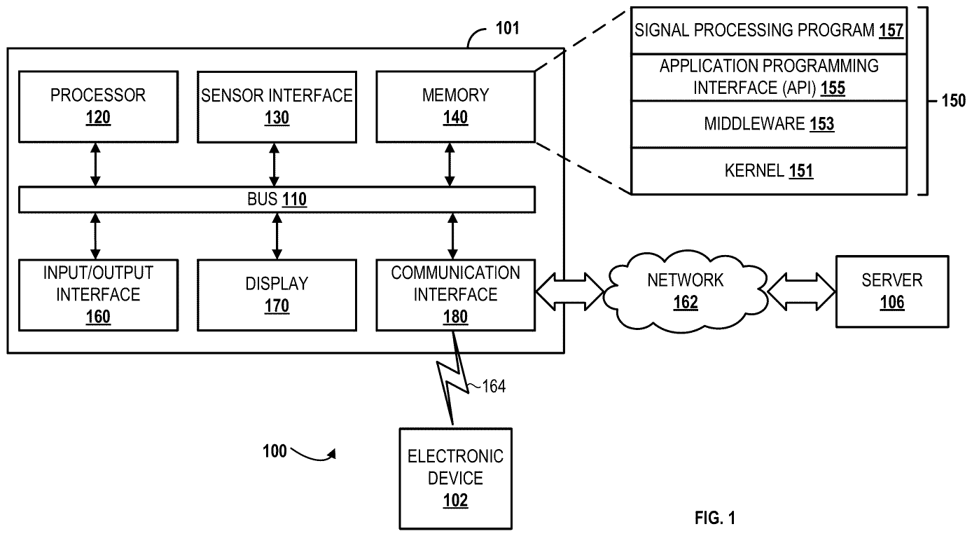

FIG. 1 shows an example system 100 for measuring tissue depth and velocity using one or more single transducers based on processing one or more ultrasound signals output by the one or more single transducers. Referring to FIG. 1, the system 100 may comprise a computing device 101. The computing device 101 may comprise, for example, a mobile phone, a smart phone, a tablet computer, a laptop, a desktop computer, a smartwatch, and the like. In an example, the computing device 101 may comprise a computing device for controlling an external device such as a medical treatment device, a powered prosthetic limb, an exoskeleton, and/or one or more human machine interfaces. As an example, the computing device 101 and the external device may be integrated together as a single device or may comprise separate devices. The computing device 101 may include a bus 110, one or more processors 120, a sensor interface 130, a memory 140, an input/output interface 160, a display 170, and a communication interface 180. In an example, the computing device 101 may omit at least one of the aforementioned constitutional elements or may additionally include other constitutional elements.

The bus 110 may include a circuit for connecting the bus 110, the one or more processors 120, the sensor interface 130, the memory 140, the input/output interface 160, the display 170, and/or the communication interface 180 to each other and for delivering communication (e.g., a control message and/or data) between the bus 110, the one or more processors 120, the sensor interface 130, the memory 140, the input/output interface 160, the display 170, and/or the communication interface 180.

The one or more processors 120 may include one or more of a Central Processing Unit (CPU), an Application Processor (AP), and a Communication Processor (CP). The one or more processors 120 may control, for example, at least one of the bus 110, the sensor interface 130, the memory 140, the input/output interface 160, the display 170, and/or the communication interface 180 and/or may execute an arithmetic operation or data processing for communication. The processing (or controlling) operation of the one or more processors 120 according to various embodiments is described in detail with reference to the following drawings.

The sensor interface 130 may comprise one or more single ultrasound transducer elements for measuring tissue depth and velocity. The single transducer elements may be positioned on one or more locations of an individual and may be configured to output one or more ultrasound signals at the one or more locations. In an example, the one or more locations may be associated with a muscle and/or tendon region of the individual. For example, the one or more locations may be associated with one or more tissues of the of the individual. As an example, the tissues of interest at each location may lie at different depths, wherein each location may comprise more than one tissue of interest (e.g., a plurality of tissues of interest). The tissues of interest at different locations may be anatomically and/or functionally synergistic (e.g., multiple muscle groups acting upon one or more joints, or blood vessels that are part of a connected vascular network). The kinematic information derived from multiple anatomical locations may be combined through additional processing to produce composite measures of physiology and function (e.g., composite measures of symmetry, timing, and propagation derived from kinematic measures at each of the one or more locations). The composite measures may utilize tissue kinematic measures in addition to other functional or physiological measures, such as accelerometry, dynamometry, force measurements, and/or electromyography. The kinematic measurements derived from one or more muscles may be utilized to determine (e.g., infer) the volitional intent of the user, e.g., the type of a specific hand grasp and its continuous proportional completion level. The one or more ultrasound signals may comprise one or more frequency-modulated continuous wave ultrasound signals. The one or more frequency-modulated continuous wave ultrasound signals may operate at a voltage between 0 and 10 volts peak-to-peak and a sweep time may comprise a duration between 1 millisecond and 100 milliseconds. As an example, the one or more ultrasound signals may comprise one or more alternating up-chirp and down-chirp waveforms that sweep between an initial frequency and a final frequency over the sweep time.

The processor-executable instructions executed by the one or more processor 120 may be stored and/or maintained by the memory 140. The memory 140 may include a volatile and/or non-volatile memory. The memory 140 may store, for example, a command or data related to at least one different constitutional element of the electronic device 101. According to various exemplary embodiments, the memory 140 may store a software and/or a program 150. The program 150 may include, for example, a kernel 151, a middleware 153, an Application Programming Interface (API) 155, and/or a signal processing program (or an “application”) 157, or the like, configured for controlling one or more functions of the computing device 101 and/or an external device. At least one part of the kernel 151, middleware 153, or API 155 may be referred to as an Operating System (OS). The memory 140 may include a computer-readable recording medium having a program recorded therein to perform the method according to various embodiments by the processor 120.

The kernel 151 may control or manage, for example, system resources (e.g., the bus 110, the processor 120, the memory 140, etc.) used to execute an operation or function implemented in other programs (e.g., the middleware 153, the API 155, or the signal processing program 157). Further, the kernel 151 may provide an interface capable of controlling or managing the system resources by accessing individual constitutional elements of the computing device 101 in the middleware 153, the API 155, or the signal processing program 157.

The middleware 153 may perform, for example, a mediation role so that the API 155 or the signal processing program 157 can communicate with the kernel 151 to exchange data. Further, the middleware 153 may handle one or more task requests received from the signal processing program 157 according to a priority. For example, the middleware 153 may assign a priority of using the system resources (e.g., the bus 110, the processor 120, or the memory 140) of the computing device 101 to at least one of the signal processing programs 157. For instance, the middleware 153 may process the one or more task requests according to the priority assigned to the at least one of the application programs, and thus may perform scheduling or load balancing on the one or more task requests.

The API 155 may include at least one interface or function (e.g., instruction), for example, for file control, window control, video processing, or character control, as an interface capable of controlling a function provided by the application 157 in the kernel 151 or the middleware 153.

The signal processing program 157 may include logic (e.g., hardware, software, firmware, etc.) that may be implemented to determine tissue depth and velocity. The signal processing program 157 may cause the computing device 101 to transmit one or more ultrasound signals via the sensor interface 130. As an example, the computing device 101 may include a programmable signal generator configured to generate the one or more ultrasound signals. The computing device 101 may receive one or more backscattered ultrasound signals based on transmitting the one or more ultrasound signals. For example, the one or more ultrasound signals may backscatter as the one or more ultrasound signals interact with an interior medium of the individual, wherein the backscattered ultrasound signals may be received by the computing device 101. The signal processing program 157 may cause the computing device 101 to generate a mixed signal based on the one or more ultrasound signals and the one or more backscattered ultrasound signals. For example, the signal processing program 157 may cause the computing device 101 to generate the mixed signal based on mixing the one or more ultrasound signals with the one or more backscattered ultrasound signals. The signal processing program 157 may cause the computing device 101 to generate an intermediate frequency signal based on applying a lowpass filter to the mixed signal.

The signal processing program 157 may cause the computing device 101 to extract range information associated with tissue depth associated with the one or more locations and kinematic (e.g., Doppler) information associated with tissue velocity associated with the one or more locations based on processing the intermediate frequency signal. For example, the signal processing program 157 may cause the computing device 101 to extract range information associated with one or more locations of different tissues and kinematic information associated with one or more locations of different tissues based on processing the intermediate frequency signal. In one example, the signal processing program 157 may cause the computing device 101 to process the intermediate frequency signal by calculating a range frequency based on adding frequency components from the up-chirp and down-chirp waveforms, and calculating a Doppler frequency based on subtracting frequency components from the up-chirp and down-chirp waveforms. In another example, the signal processing program 157 may cause the computing device 101 to process the intermediate frequency signal by reorganizing the intermediate frequency signal into a time-windowed two-dimensional array comprising fast time samples and slow time samples, calculating a range frequency based on analyzing the fast time samples of the time-windowed two-dimensional array, and calculating a Doppler frequency based on analyzing the slow time samples of the time-windowed two-dimensional array. The analysis steps may be computed in the frequency domain using a two-dimensional Fourier Transform or in the time domain using a two-dimensional autocorrelation function. The range information may be determined from the range frequency according to a relationship between bandwidth, the sweep time, and a speed of sound in tissue. The Doppler information may be determined from the Doppler frequency according to a relationship between the initial frequency and the speed of sound in tissue.

The signal processing program 157 may cause the computing device 101 to perform one or more actions based on extracting the range information and the kinematic information. As one example, the signal processing program 157 may cause the computing device 101 to generate one or more physiological measurements associated with one or more areas of the individual based on the range information associated with the one or more locations and the kinematic information associated with the one or more locations. The one or more physiological measurements may comprise one or more of muscle and displacement, muscle contraction velocities, tendon velocities, muscle and tendon strain, blood flow velocities, artery wall velocities and displacement, artery wall blood pressure pulse wave velocity, lung motion, cardiac kinematics and blood flow, brain tissue motion and blood flow, or other tissue kinematics.

As another example, the signal processing program 157 may cause the computing device 101 to determine one or more medical conditions associated with the individual. The one or more medical conditions may comprise one or more of one or more cardiovascular conditions, peripheral vascular conditions, cerebrovascular conditions, or musculoskeletal conditions. The signal processing program 157 may cause the computing device 101 to output (e.g., display) one or more notifications associated with the one or more medical conditions. The signal processing program 157 may cause the computing device 101 to cause one or more treatments associated with the individual based on the one or more medical conditions. For example, the computing device 101 may be configured with a medical treatment device for administering the one or more treatments. For example, the computing device 101 may control a medical treatment device to administer the one or more treatments. In an example, the signal processing program 157 may cause the computing device 101 to determine a response of the individual to a medical treatment or a medical procedure based on one or more values of the range information and one or more values of the kinematic information, and output one or more notifications associated with the response of the individual to the medical treatment or the medical procedure. For example, the one or more values of the range information and the one or more values of the kinematic information may comprise current and historical values of the range information and the kinematic information, respectively. In an example, the signal processing program 157 may cause the computing device 101 to output a measure of functional performance of the individual based on the range information and the kinematic information. In an example, the signal processing program 157 may cause the computing device 101 to output a measure of recovery status of the individual after an injury based on the range information and the kinematic information.

As another example, the signal processing program 157 may cause the computing device 101 to output one or more signals for controlling an external device based on the range information and the kinematic information. For example, the external device may comprise a powered prosthetic limb, an exoskeleton, or one or more human machine interfaces. The one or more human machine interfaces may comprise one or more of feedback for neurostimulation, neuromuscular electrical stimulation, or functional electrical stimulation.

As another example, the signal processing program 157 may cause the computing device 101 to generate image data associated with the one or more locations on the individual. For example, the computing device 101 may output (e.g., display via display 170) the image data. The image data may comprise one or more M-mode ultrasound images indicating locations of tissue interfaces over time or one or more M-mode ultrasound images indicating kinematics of tissue interfaces (including displacement, velocity, acceleration, jerk, strain, and strain rate) over time.

The input/output interface 160 may be configured as an interface for delivering an instruction or data input from a user or a different external device(s) to the processor 120, the memory 140, the display 170, and the communication interface 180. For example, the input/output interface 160 may receive user input for programming the computing device 101. For example, the input/output interface 160 may receive user input adjusting one or more settings of the computing device 101, and/or settings of one or more of the external devices such as the powered prosthetic limb, the exoskeleton, or the one or more human machine interfaces). Further, the input/output interface 160 may output an instruction or data received from the processor 120, the sensor interface 130, the memory 140, the input/output interface 160, the display 170, and/or the communication interface 180 to a different external device (e.g., electronic device 102, server 106, etc.).

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body. As an example, the display 170 may output a user interface configured to display the image data. In an example, the display 170 may display a visual notification of, or associated with, the one or more physiological measurements and/or the one or more medical conditions.

The communication interface 180 may establish, for example, communication between the computing device 101 and an external device (e.g., an electronic device 102 or one or more servers 106). For example, the communication interface 180 may communicate with the external device (e.g., the electronic device 102 or the one or more servers 106) by being connected to a network 162 through wireless communication or wired communication. For example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the network 162 may include at least one of a telecommunications network, a computer network (e.g., LAN or WAN), the internet, and a telephone network.

In addition, the communication interface 180 may communicate with the external device (e.g., the electronic device 102) via a communication connection 164 such as a wireless communication and/or wired communication. The wireless communication may include, for example, a near-distance communication 164. The near-distance communications 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like.

The server 106 may include a group of one or more servers. In an example, all or some of the operations executed by the computing device 101 may be executed in a different one or a plurality of electronic devices (e.g., the electronic device 102 and/or the one or more servers 106). In an example, if the computing device 101 needs to perform a certain function or service either automatically or based on a request, the computing device 101 may request at least some parts of functions related thereto alternatively or additionally to a different electronic device (e.g., the electronic device 102 and/or the server 106) instead of executing the function or the service autonomously. The different electronic devices (e.g., the electronic device 102 and/or the server 106) may execute the requested function or additional function, and may deliver a result thereof to the computing device 101. In an example, the electronic device 102 may comprise a mobile phone, a smart phone, a tablet computer, a laptop, a desktop computer, a smartwatch, and the like. The electronic device 102 may be configured to process the one or more ultrasound signals and/or the one or more backscattered ultrasound signals. For example, the electronic device 102 may be configured to receive the one or more ultrasound signals and/or the one or more backscattered ultrasound signals from the computing device 101, wherein the electronic device 102 may process the one or more ultrasound signals and/or the one or more backscattered ultrasound signals to determine the range information and the kinematic information. As a further example, one or more of the servers 106 may be configured to receive the one or more ultrasound signals and/or the one or more backscattered ultrasound signals from the computing device 101 and process the one or more ultrasound signals and/or the one or more backscattered ultrasound signals to determine the range information and the kinematic information. The computing device 101 may provide the requested function or service either directly or by additionally processing the received result. For example, a cloud computing, distributed computing, or client-server computing technique may be used.

Each of the constitutional elements described in the present document may consist of one or more components, and names thereof may vary depending on a type of an electronic device. The computing device 101 may include at least one of the constitutional elements described in the present document. Some of the constitutional elements may be omitted, or additional other constitutional elements may be further included. Further, some of the constitutional elements of the computing device 101 according to various exemplary embodiments may be combined and constructed as one entity, so as to equally perform functions of corresponding constitutional elements before combination.

The system 100 described in FIG. 1 may represent a technical improvement to several technical problems associated with conventional ultrasound systems. Conventional ultrasound systems typically require high-voltage transmission signals ranging from 20 to 100 volts peak-to-peak and short-duration pulses, necessitating specialized front-end electronics, transmit/receive switches, and high-speed digitization systems operating at 20 to 100 mega-samples per second. These hardware demands create substantial constraints on miniaturization and power consumption. In contrast, the system 100 may operate using frequency-modulated continuous wave ultrasound signals at voltages between 0 and 10 volts peak-to-peak, which may reduce power consumption and simplify hardware requirements. Additionally, conventional Doppler ultrasound systems typically lack the ability to simultaneously measure both tissue depth and velocity using a single transducer element, often requiring separate transmit and receive transducers positioned at predetermined angles. The system 100 may address this limitation by enabling simultaneous extraction of range information and Doppler information from a single transducer element through processing of the intermediate frequency signal. This approach may eliminate the need for complex transducer arrays and may reduce placement sensitivity, potentially enabling wearable implementations for continuous monitoring during dynamic activities. Furthermore, the system 100 may provide depth-resolved velocity measurements without requiring precise transducer positioning by trained personnel, which may expand accessibility of ultrasound-based monitoring beyond specialized clinical environments.

As an example, the signal processing program 157 may cause the computing device 101 to process the one or more ultrasound signals based on implementing a Time-Delayed Spectroscopy-enabled Doppler (TDS-D) process that comprises a low-voltage, long-duration, frequency-modulated ultrasound Doppler measurement process. For example, the TDS-D process may simultaneously measure tissue motion velocity and depth using a single transducer. As an example, the system 100 may be configured to monitor tissue interfaces during exercise, providing real-time data on tissue depth and kinematics in a wearable form factor, while individuals perform dynamic motion tasks. Furthermore, the TDS-D may be utilized to enable quantitative measurement of muscle and tendon velocities, providing a physiological biomarker during functional tasks. For example, the system 100 may be configured to enable real-time monitoring of internal bodily functions during dynamic activity. In an example, system 100 may also be configured to quantify blood flow velocities or motion of artery walls. For example, the continuous measurement of the motion of the artery walls can be related to continuous blood pressure.

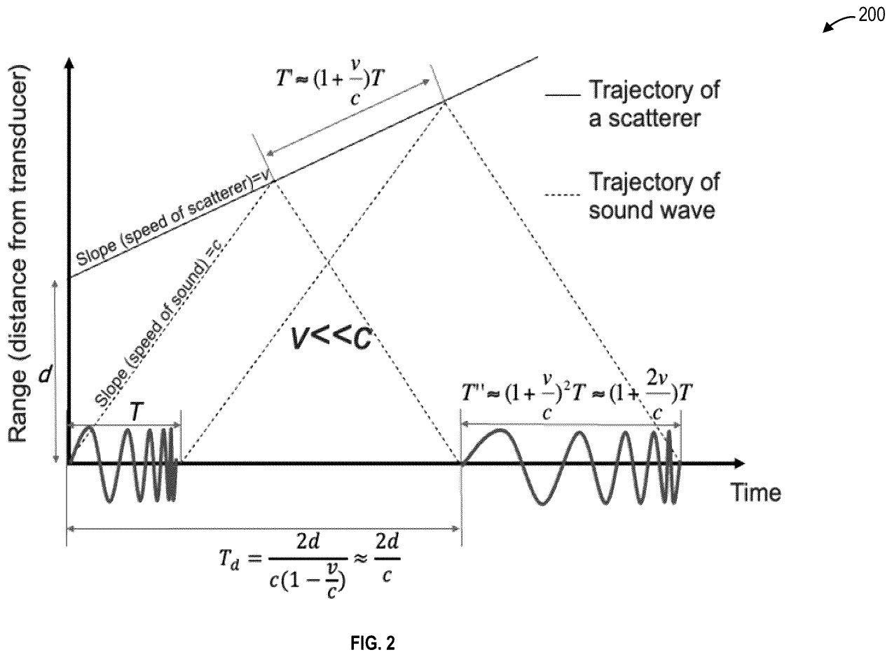

As shown in FIG. 2, an ultrasound signal comprising a frequency modulated signal may be transmitted by an ultrasound transducer at time t=0 interacting with a scatterer at an initial depth of d. The ultrasound signal travels at the speed of sound, c, while the scatter travels at a speed, v away from the transducer. As a result of the interaction, the backscattered reflected signal is shifted in time by T_{d}, and its time duration is expanded from T to T″, caused by the interaction between the traveling ultrasound wave and the moving scatterer, leading to a Doppler shift in the instantaneous frequency. For example, a frequency modulated signal may be transmitted by the ultrasound transducer, where

$${x}_{T} ( t ) = {A}_{T} cos ( 2 πφ ( t ) ) , \qquad ( 1 )$$

where A_{T} is the transmitted amplitude and φ(t) is the instantaneous phase. The instantaneous frequency ƒ_{T}(t) is given by the derivative of the instantaneous phase,

$${f}_{T} ( t ) = \frac{d φ ( t )}{dt} = {f}_{0} + B {f}_{m} ( t ) , \qquad ( 2 )$$

where ƒ₀ is the initial frequency and ƒₘ(t) is a slow-varying modulation function. B is an adjustable factor that represents the degree of modulation. For example, when B=0, there is no frequency modulation.

The modulation function ƒₘ(t) may be chosen to have different properties. For example, the modulation function may be a sinusoid with a frequency of ƒₘ.

$${f}_{m} ( t ) = sin ( 2 π {f}_{m} t ) , \qquad ( 3 )$$

In an example, the modulation function may have the form of a sawtooth wave, where the frequency increases linearly during a period Tₛ, where

$${f}_{m} ( t ) = \vert \frac{t}{{T}_{s}} - ⌊ \frac{t}{{T}_{s}} ⌋ \vert , \qquad ( 4 )$$

$$where ⌊ \frac{t}{{T}_{s}} ⌋$$

where represents the floor function.

In an example, the modulation function ƒₘ(t) may comprise a triangular wave, where the frequency increases linearly and then decreases linearly. For example, the function ƒₘ(t) may be represented as

$${f}_{m} ( t ) = 2 \vert \frac{t}{2 {T}_{s}} - ⌊ \frac{t}{2 {T}_{s}} + \frac{1}{2} ⌋ \vert . \qquad ( 5 )$$

As an example, a triangular modulation function may be considered, with the instantaneous frequency as a linear chirp increasing from an initial frequency of ƒ₀ to a frequency of ƒₑ (e.g., the up-chirp), and then decreasing linearly back to ƒ₀ (e.g., the down-chirp). In this case, the modulation factor B comprises the bandwidth B=ƒₑ−ƒ₀, and Tₛ comprises the duration of each up and down chirp sweep (e.g., the sweep time). The ratio

$$\frac{B}{{T}_{s}}$$

comprises the sweep rate.

As the acoustic wave travels within a medium (e.g., an interior of an individual) and interacts with a scatterer ultrasound signal at a distance d from the transducer moving at a constant velocity v, the received backscattered ultrasound signal with amplitude A_{R} is delayed in time, and the instantaneous frequency is Doppler shifted, as shown in FIG. 2. As such, the received signal may be represented as

$${y}_{R} ( t ) = {A}_{R} cos ( 2 π θ ( t ) ) , where \qquad ( 6 )$$

$${f}_{R} ( t ) = \frac{dθ ( t )}{dt} = \frac{{f}_{T} ( t - \frac{2 d}{c} )}{{( 1 + \frac{v}{c} )}^{2}} \approx ( 1 - \frac{2 v}{c} ) {f}_{T} ( t - \frac{2 d}{c} ) = {f}_{T} ( t - \frac{2 d}{c} ) - \frac{2 v}{c} {f}_{T} ( t - \frac{2 d}{c} ) . \qquad ( 7 )$$

The transmitted and received signals can then be mixed together to yield

$${x}_{T} ( t ) {y}_{R} ( t ) = {A}_{T} {A}_{R} cos ( 2 πφ ( t ) ) cos ( 2 π θ ( t ) ) = \frac{1}{2} {A}_{T} {A}_{R} [ cos ( 2 π ( φ ( t ) - θ ( t ) ) ) + cos ( 2 π ( φ ( t ) + θ ( t ) ) ) ] . \qquad ( 8 )$$

A low pass filter is then used to isolate a difference frequency and suppress the higher sum frequency resulting in an intermediate frequency signal,

$$IF ( t ) = A cos ( 2 π ( φ ( t ) - θ ( t ) ) ) = A cos ( 2 π ( {\int }_{0}^{t} [ {f}_{T} ( τ ) - {f}_{R} ( τ ) ] d τ ) ) . \qquad ( 9 )$$

Substituting from equation 7,

$${f}_{T} ( t ) - {f}_{R} ( t ) = {f}_{T} ( t ) - {f}_{T} ( t - \frac{2 d}{c} ) + \frac{2 v}{c} {f}_{T} ( t - \frac{2 d}{c} ) . \qquad ( 10 )$$

Substituting from equation 2,

$${f}_{T} ( t ) - {f}_{R} ( t ) = B [ {f}_{m} ( t ) - {f}_{m} ( t - \frac{2 d}{c} ) ] + \frac{2 v}{c} [ {f}_{0} + B {f}_{m} ( t - \frac{2 d}{c} ) ] . \qquad ( 11 )$$

In an example, considering a sawtooth modulation function in equation 5, with a periodically repeating up chirp, in consideration of the up chirps separately for the time interval [0, Tₛ], for the up chirp in this interval,

$${f}_{m} ( t ) = \frac{t}{{T}_{s}} .$$

Substituting into equation 9 and equation 11,

$${IF}_{up} ( t ) = Acos ( 2 π ( {\int }_{0}^{t} B [ \frac{2 d}{{cT}_{s}} ] d τ + {\int }_{0}^{t} \frac{2 v}{c} [ {f}_{0} + \frac{B}{{T}_{s}} ( τ - \frac{2 d}{c} ) ] d τ ) ) . \qquad ( 12 )$$

If d and v are assumed to be constants, after performing the integration, equation 12 simplifies to

$${IF}_{up} ( t ) = Acos ( 2 π [ ( \frac{2 {vf}_{0}}{c} + \frac{2 B}{{T}_{s}} \frac{d}{c} ( 1 - \frac{2 v}{c} ) ) t + \frac{B}{{T}_{s}} ( \frac{v}{c} ) {t}^{2} ] ) . \qquad ( 13 )$$

The demodulated intermediate frequency signals contain the range frequency,

$$\frac{2 Bd}{{T}_{s} c} ,$$

and doppler shift frequencies

$$\frac{2 {vf}_{0}}{c} ,$$

and the frequency varies with a sweep rate of

$$\frac{B}{{T}_{s}} ( \frac{v}{c} ) .$$

It can be seen from equation 13 that if the scatterer velocity (v) is zero, then the intermediate frequency signal involves a range frequency shift

$${f}_{range} = \frac{2 B}{{T}_{s}} \frac{d}{c}$$

corresponding to the range of the scatterer d, where

$${IF}_{up} ( t ) = Acos ( 2 π [ ( \frac{2 B}{{T}_{s}} \frac{d}{c} ) t ] ) . \qquad ( 14 )$$

Conversely, if there is no frequency modulation, such as, if B=0, then the intermediate frequency signal involves a Doppler frequency shift

$${f}_{dop} = \frac{2 {vf}_{0}}{c}$$

corresponding to the velocity of the scatterer v, which is the case of continuous wave Doppler, where

$${IF}_{up} ( t ) = Acos ( 2 π [ ( \frac{2 {vf}_{0}}{c} ) t ] ) . \qquad ( 15 )$$

For a triangular modulation function with periodically repeating up and down chirps, the expression for the intermediate frequency resulting from the up chirp remains the same. The corresponding down chirp in the interval, [0, Tₛ], may be obtained by substituting

$${f}_{m} ( t ) = 1 - \frac{t}{{T}_{s}}$$

in equation 11, where

$${IF}_{down} ( t ) = ( 2 π [ ( \frac{2 {vf}_{e}}{c} - \frac{2 B}{{T}_{s}} \frac{d}{c} ( 1 + \frac{2 v}{c} ) ) t - \frac{B}{{T}_{s}} ( \frac{v}{c} ) {t}^{2} ] ) . \qquad ( 16 )$$

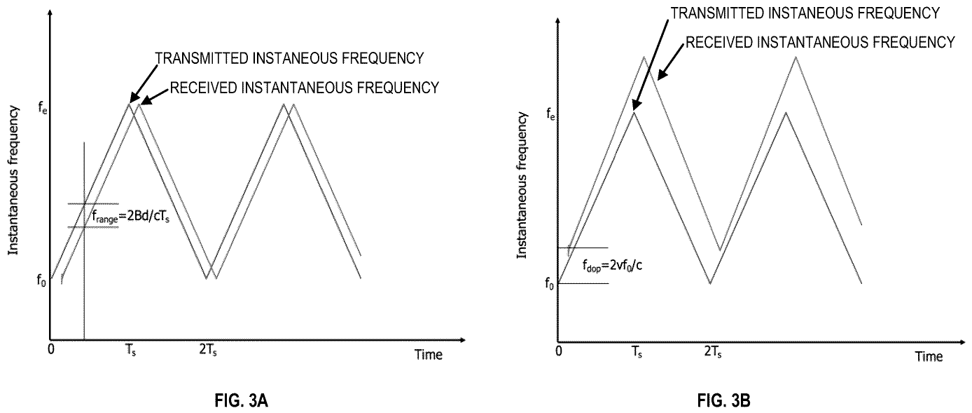

The range frequency shift has a negative sign relative to the up chirp in equation 13, while the Doppler frequency shift has a positive sign. FIGS. 3A-3B show a graphical visualization of the transmitted and received instantaneous frequencies. FIG. 3A shows the instantaneous transmit ƒ_{T}(t) and receive frequencies ƒ_{R}(t) for a triangular chirp signal (e.g., linear up and down chirp) in the case of a stationary scatterer at a depth d. The received signal is shifted in time, which corresponds to a frequency shift ƒ_{range}. FIG. 3B shows the instantaneous transmit and receive frequencies for a triangular chirp signal (e.g., linear up and down chirp) in the case of a scatterer at a depth d moving with a speed v. The received signal is shifted in time as well as Doppler shifted with frequency shift ƒ_{d}op.

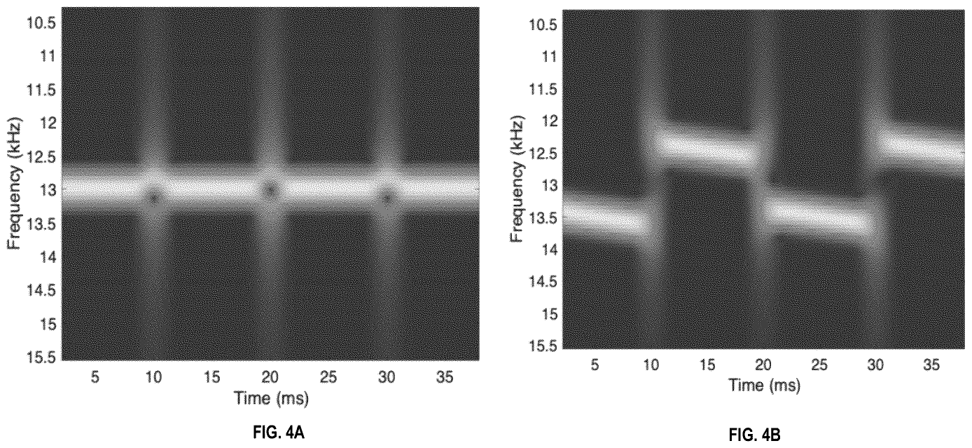

FIGS. 4A-4B show a simulated spectrogram of the intermediate frequency signal for a triangular chirp signal (e.g., linear up and down chirp) with f₀=3 MHz, B=2 MHz, and Tₛ=10 ms. FIG. 4A shows the simulated spectrogram for a stationary scatterer at a depth 5 cm, with a corresponding range shift of ƒ_{range}≈13 kHz. FIG. 4B shows the simulated spectrogram for a stationary scatterer at a depth 5 cm moving with a speed 10 cm/s away from the transducer. In this case, the range shift is ƒ_{range}≈13 kHz and the Doppler shift is ƒ_{dop}≈390 Hz. As shown in FIGS. 4A-4B the range shift and the Doppler shift may have different effects on the intermediate frequency signal for the up and down chirps. In an example, the Doppler shift also introduces a frequency modulation of the intermediate frequency signal with a slope

$$\frac{B}{{T}_{s}} ( \frac{v}{c} ) .$$

Different sweep rates may further differentiate between the range frequency shift and the Doppler frequency shift. In an example, the range resolution depends on the sweep rate

$$\frac{B}{{T}_{s}} ,$$

with increasing sweep rates enabling better frequency separation between two interfaces separated by a fixed distance. The Doppler resolution depends on the initial chirp frequency. The pulse duration Tₛ also determines the ability to resolve frequency shifts because of the time-bandwidth product. Longer duration pulses have better frequency resolution both for range and Doppler shift.

These methods may be utilized to separate the range and Doppler shift frequencies using digital signal processing. For example, slow and fast movements may be analyzed. For example, motion in a human body occurs at a much smaller speed (e.g., blood flow is less than 1 m/s while tissue motion is less than 10 cm/s) compared to the speed of sound in tissue (e.g., 1540 m/s). Thus, for a starting chirp frequency of 3 MHz, the Doppler shift frequency is less than 3 kHz for blood flow and may be less than 300 Hz for tissue motion. In contrast, the frequency shift corresponding to the range,

$$\frac{2 Bd}{{T}_{s} c} ,$$

depends on the choice of the sweep rate and the range of the scatterer of interest. For example, with a 3-5 MHz sweep over 10 ms, the range frequency shift for a scatterer at 5 cm depth is ~13 kHz. As such, for tissue motion, the range frequency shift is often an order of magnitude higher than the Doppler frequency shift. The analysis can be performed in the frequency domain or the time domain using different approaches for different embodiments.

As an example, the frequency domain may be analyzed using a two-dimensional (2D) power spectral density of the intermediate signal. For example, the transmitted ultrasound signal may be frequency modulated with a periodic function with period of Tₛ. For example, a triangular modulating waveform may be utilized such that the transmitted signal consists of a linear up chirp from frequency ƒ₀ to ƒₑ over Tₛ seconds, and a linear down chirp from frequency ƒₑ to ƒ₀ over Tₛ seconds. For example, the received demodulated intermediate frequency signal IF(t) may be digitized at a rate Fᵣ. For example, a sample may be obtained every

$${T}_{r} = \frac{1}{{F}_{r}}$$

seconds. In an example, a scatterer at depth d, may be moving at a velocity v. In the human body, the scatterer movement is expected to be time varying, so both the instantaneous depth and velocity are functions of time. Thus, from equation 12,

$${IF}_{up} ( t ) = Acos ( 2 π ( \frac{2 B}{{cT}_{s}} {\int }_{0}^{t} d ( τ ) d τ + \frac{2 {f}_{0}}{c} {\int }_{0}^{t} v ( τ ) d τ + \frac{2 B}{{cT}_{s}} {\int }_{0}^{t} τ v ( τ ) d τ - \frac{4 B}{{c}^{2} {T}_{s}} {\int }_{0}^{t} d ( τ ) v ( τ ) d τ ) ) . \qquad ( 17 )$$

The factor

$$\frac{4 B}{{c}^{2} {T}_{s}}$$

is very small, so the expression can be simplified to

$${IF}_{up} ( t ) = Acos ( 2 π ( \frac{2 {f}_{0}}{c} {\int }_{0}^{t} v ( τ ) d τ + \frac{2 B}{c {T}_{s}} {\int }_{0}^{c} [ d ( τ ) + τ v ( τ ) ] d τ ) ) . \qquad ( 18 )$$

In an example, a similar expression can be derived for the down chirp, where

$${IF}_{down} ( t ) = Acos ( 2 π ( \frac{{f}_{0}}{c} {\int }_{0}^{c} v ( τ ) d τ - \frac{2 B}{c {T}_{s}} {\int }_{0}^{c} [ d ( τ ) + τ v ( τ ) ] d τ ) ) . \qquad ( 19 )$$

The digitized signal may be reorganized into a 2D signal consisting of a set of fast time samples obtained at times, nTᵣ, that denotes time within the duration of the chirp, and a set of slow time samples obtained at times, kTₛ, that denotes the samples between multiple received chirp signals.

In an example, a simplifying assumption may be made that the scatterer position d(t) and velocity v(t) are slowly varying relative to the duration of the chirp Tₛ. Thus, in fast time, the position and velocity can be considered to be constant d(kTₛ) and v(kTₛ) within the duration of the chirp, where

$$I ( n , k ) = {IF}_{up} ( {nT}_{r} , {kT}_{s} ) = Acos ( 2 π [ ( \frac{2 v ( {kT}_{s} ) {f}_{0}}{c} ) {nT}_{r} + ( \frac{2 B}{{T}_{s}} \frac{d ( {kT}_{s} )}{c} ) {nT}_{r} + \frac{Bv ( {kT}_{s} )}{{cT}_{s}} {( {nT}_{r} )}^{2} ] ) . \qquad ( 20 )$$

The 2D power spectral density of a windowed portion of this demodulated intermediate frequency signal can be computed using the 2D Fourier Transform, where

$${Φ}_{up} ( {F}_{fast} , {F}_{slow} ) = \vert ℱ ( l ( n , k ) * W ( n , k ) \vert , \qquad ( 21 )$$

where W is a window function used to select the portion of the intermediate frequency signal for analysis. The choice of W also impacts the range and Doppler resolution, since it shapes the 2D power spectral density.

The spectral centroids of the 2D power spectral density are given by

$$〈 {f}_{dop} 〉 = \frac{\int {\int }_{- 0.5}^{0.5} {F}_{slow} {Φ}_{up} ( {F}_{fast} , {F}_{slow} ) {dF}_{fast} {F}_{slow}}{\int {\int }_{- 0.5}^{0.5} {Φ}_{up} ( {F}_{fast} , {F}_{slow} ) {dF}_{fast} {F}_{slow}} ; \qquad ( 22 )$$

$$〈 {f}_{range} 〉 = \frac{\int {\int }_{- 0.5}^{0.5} {F}_{fast} {Φ}_{up} ( {F}_{fast} , {F}_{slow} ) {dF}_{fast} {F}_{slow}}{\int {\int }_{- 0.5}^{0.5} {Φ}_{up} ( {F}_{fast} , {F}_{slow} ) {dF}_{fast} {F}_{slow}} . \qquad ( 23 )$$

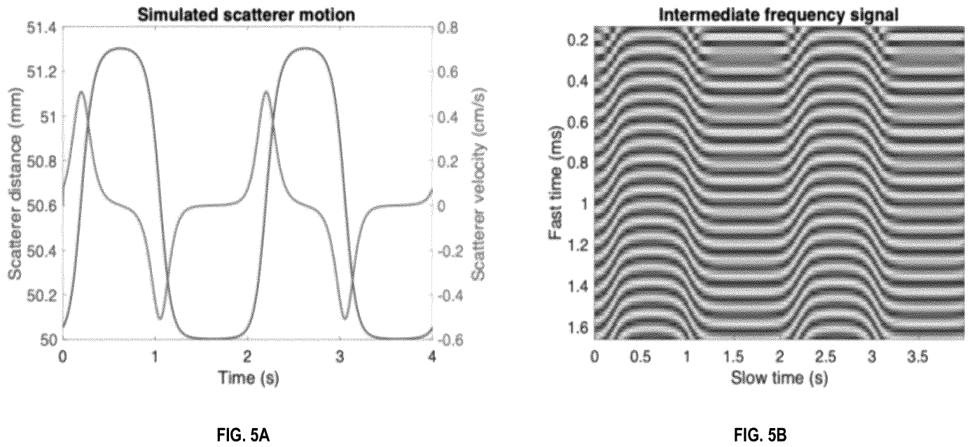

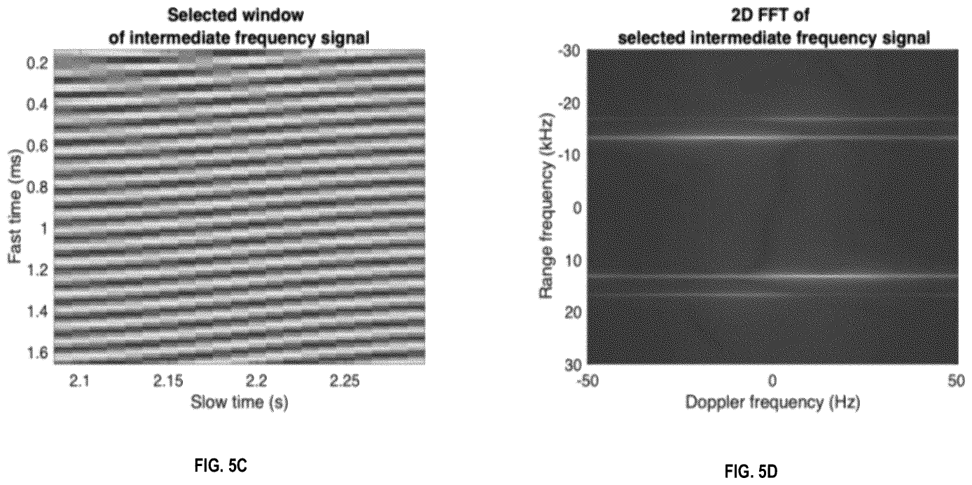

FIGS. 5A-5D show example data associated with the process of analyzing the frequency domain using the 2D power spectral density of the intermediate frequency signal. For example, a signal processing algorithm may be used for estimating time varying scatter motion. FIG. 5A shows a simulated scatterer at an initial depth of 50 mm, moving periodically every two seconds up to a depth of 51.3 mm. The maximum scatterer velocity is +/−0.5 cm/s. This simulation mimics a scattering interface between muscle and fascia while the muscle is contracting and relaxing every two seconds. FIG. 5B shows the scatterer imaged using a triangular chirp signal with f₀=3 MHz, B=2 MHz, and Tₛ=10 ms. The intermediate frequency signal can be represented as a 2D matrix, where the values in the rows of each column correspond to the samples of the demodulated signal within each chirp (e.g., fast time), and each column corresponds to consecutive chirps (e.g., slow time). FIG. 5C shows that a selected fast and slow time window of the intermediate frequency signal shows the variation in slow time corresponds to the maximum positive velocity of the scatterer. FIG. 5D shows that the corresponding 2D Fourier Transform of the 2D signal shows the Doppler frequency axis (e.g., corresponding to slow time) and the Range frequency axis (e.g., corresponding to fast time). In this case, the range shift frequency is ~13.3 kHz, and the Doppler shift frequency is ~30 Hz. As an example, due to time-varying velocity within the analysis window, there is a spreading of the Doppler spectrum. The Doppler spectrum can be estimated by taking the projection along all range frequencies, while the range spectrum can be estimated by taking the projection along all the Doppler frequencies. Frequency domain filters can be applied before taking the projection to select specific range and Doppler frequency shifts of interest. The mean frequency shift (e.g., the tissue or blood flow velocity) can be estimated from the spectral centroid of the Doppler spectrum.

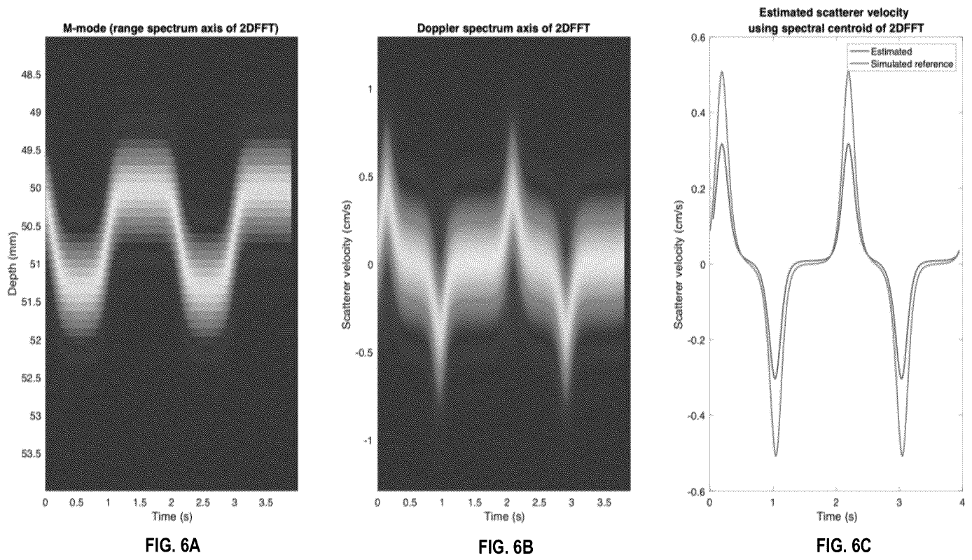

FIGS. 6A-6C show examples of data associated with the power spectral density. FIG. 6A shows that the power spectral density spectrogram of the range frequency shift is reconstructed as an ultrasound M-mode image showing the scatterer position over time. FIG. 6B shows that the corresponding power spectral density spectrogram of the Doppler frequency shift estimated from the 2D FFT is reconstructed as an ultrasound Doppler spectrogram showing the scatterer velocity over time. The Doppler frequency shifts over time follows the waveform of the simulated scatterer velocity with a processing latency corresponding to the Fourier analysis window. FIG. 6C shows the scatterer velocity estimated using the spectral centroid of the 2D FFT. The estimated velocity waveform matches the waveform of the simulated scatterer velocity with a processing latency corresponding to the autocorrelation analysis window.

As an example, the following steps of the frequency domain signal processing algorithm may be implemented:
1. The transmitted frequency modulated ultrasound signal x_{R}(t), with a periodic modulation function, is mixed with the amplified received backscattered ultrasound signal y_{R}(t).
2. A lowpass filter is applied to the mixed signal to yield a downmixed intermediate frequency signal IF(t), that consists of lower frequency components in the audio frequency range representing both range shift frequencies corresponding to scatterers at different depths, as well as Doppler shift frequencies corresponding to the velocity of each scatterer.
3. The digitized intermediate frequency signal is reorganized into fast and slow time samples to yield a 2D signal IF(nTᵣ,kTₛ), where the signal is sampled at two rates: a fast time sampling rate yielding samples every Tᵣ seconds, and a slow time sampling rate yielding samples every Tₛ seconds, where Tₛ is an integer multiple of Tᵣ, and Tₛ is chosen to be the period of the periodic modulation function.
4. The digitized 2D signal is windowed using a 2D window function to select a fast and slow time analysis window. The 2D signal can be separated into separate analysis windows corresponding to the periodic modulation function. For example, for a triangular modulation function, the up and down chirps can be analyzed separately. For a non-uniform modulation function (e.g., an exponentially decaying chirp), different time windows of the modulated wave may be analyzed separately.
5. The 2D power spectral density of the windowed 2D signal is computed. Different algorithms can be used to compute the power spectral density. These include the 2DFFT algorithm, the Welch's periodogram method, as well as high resolution parametric methods that include Burg's method, or MUSIC or ESPRIT methods.
6. The 2D power spectral density is optionally filtered in the frequency domain to select frequency shifts of interest.
7. The range shift spectra and the Doppler shift spectra are extracted from the 2D power spectral density for each analysis window by taking suitable projections along each frequency axis.
8. The range shift spectra for each slow time analysis window produces the M-mode ultrasound image that represents the brightness and range of each scattering interface as a function of slow time.
9. The spectral centroid of the Doppler shift spectrum at each depth can be used to create a M-mode velocity image.
10. The spectral power of the Doppler shift spectrum at each depth can be used to create a M-mode power Doppler image.
11. The M-mode power Doppler image can be used to identify the moving scatterers with the highest Doppler power, and their corresponding velocities can be extracted from the M-mode velocity image.

As an example, the time domain may be analyzed using a 2D autocorrelation of the analytical intermediate frequency signal. In an example, the computations may be implemented in the time domain using the 2D autocorrelation applied to the reorganized digitized analytical intermediate frequency signal. In an example, the analytical signal [symbol] (n,k) corresponding to the intermediate frequency signal IF(n,k) may be obtained using the Hilbert transform along the fast time direction. The 2D autocorrelation [symbol] (n′,k′) is then defined by

$$\text{?} ( {n}^{′} , {k}^{′} ) = {\sum }_{k} {\sum }_{n} \text{?} ( n , k ) * {\text{?}}^{*} ( n + {n}^{′} , k + {k}^{′} ) . \qquad ( 24 )$$

The 2D autocorrelation function [symbol] (n′,k′) is also the inverse Fourier transform of the 2D power spectrum. The discrete derivatives of the 2D autocorrelation function can be shown to correspond to the spectral centroids of the 2D power spectrum, where

$$〈 {f}_{dop} 〉 = \frac{{\frac{- j \partial }{2 π} \frac{\text{?} ( {n}^{′} , {k}^{′} )}{2 {k}^{′}} \vert}_{{n}^{′} = {k}^{′} = 0}}{\text{?} ( 0 , 0 )} \approx \frac{1}{2 π} {tan}^{- 1} \{ \frac{Im [ \text{?} ( 0 , 1 ) ]}{Re [ \text{?} ( 0 , 1 ) ]} \} , \qquad ( 25 )$$

$$and$$

$$〈 {f}_{range} 〉 = \frac{{\frac{- j \partial }{2 π} \frac{\text{?} ( {n}^{′} , {k}^{′} )}{2 {k}^{′}} \vert}_{{n}^{′} = {k}^{′} = 0}}{\text{?} ( 0 , 0 )} \approx \frac{1}{2 π} {tan}^{- 1} \{ \frac{Im [ \text{?} ( 0 , 1 ) ]}{Re [ \text{?} ( 0 , 1 ) ]} \} . \qquad ( 26 )$$

The estimated velocity can then be derived using

$$〈 v 〉 \approx \frac{c}{2 {πf}_{0} {T}_{s}} \frac{〈 {f}_{dop} 〉}{〈 {f}_{range} 〉} . \qquad ( 27 )$$

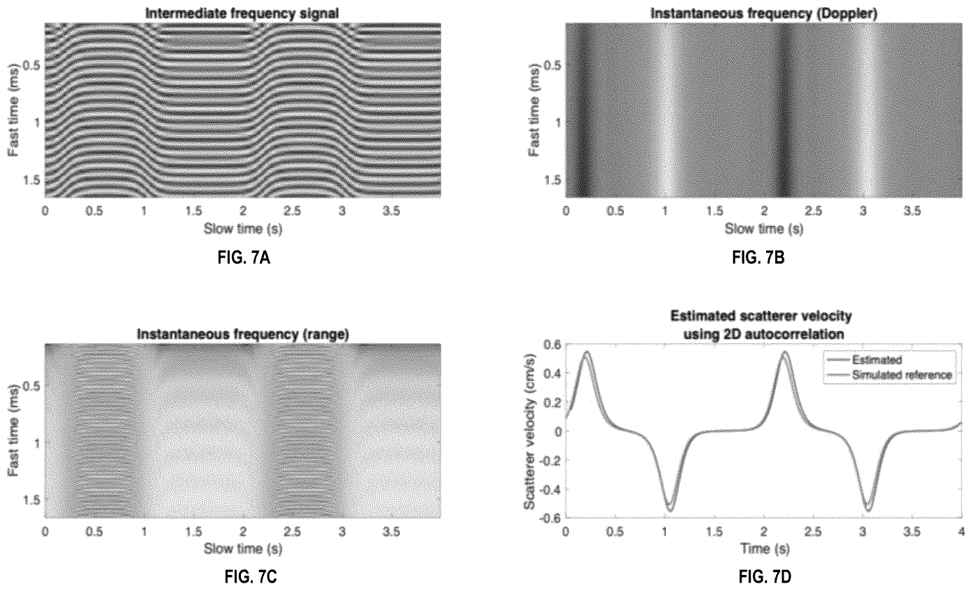

FIGS. 7A-7C show examples of data associated with the intermediate frequency signal. FIG. 7A shows that the intermediate frequency signal can be represented as a 2D matrix, where the values in the rows of each column correspond to the samples of the demodulated signal within each chirp (e.g., fast time), and each column corresponds to consecutive chirps (e.g., slow time). FIG. 7B shows the corresponding instantaneous Doppler frequency shift estimated from the 2D autocorrelation function from equation 25. FIG. 7C shows the corresponding instantaneous Doppler frequency shift estimated from the 2D autocorrelation function from equation 26. FIG. 7D shows the scatterer velocity estimated using the lags of the 2D autocorrelation using equation 27. The estimated velocity waveform closely matches the waveform of the simulated scatterer velocity with a processing latency corresponding to the autocorrelation analysis window.

As an example, the following steps of the time domain signal processing algorithm may be implemented:
1. The transmitted frequency modulated ultrasound signal x_{R}(t), with a periodic modulation function, is mixed with the amplified received backscattered ultrasound signal y_{R}(t).
2. A lowpass filter is applied to the mixed signal to yield a downmixed intermediate frequency signal IF(t), that consists of lower frequency components in the audio frequency range representing both range shift frequencies corresponding to scatterers at different depths, as well as Doppler shift frequencies corresponding to the velocity of each scatterer.
3. The digitized intermediate frequency signal is reorganized into fast and slow time samples to yield a 2D signal IF(nTᵣ,kTₛ), where the signal is sampled at two rates: a fast time sampling rate yielding samples every Tᵣ seconds, and a slow time sampling rate yielding samples every Tₛ seconds, where Tₛ is an integer multiple of Tᵣ, and Tₛ is chosen to be the period of the periodic modulation function.
4. The digitized 2D signal is windowed using a 2D window function to select a fast and slow time analysis window. The 2D signal can be separated into separate analysis windows corresponding to the periodic modulation function. For example, for a triangular modulation function, the up chirp and down chirps can be analyzed separately. For a non-uniform modulation function (e.g., an exponentially decaying chirp), different time windows of the modulated wave may be analyzed separately.
5. The analytical form of the digitized 2D intermediate frequency signal is obtained using the Hilbert transform.
6. The 2D autocorrelation of the windowed 2D signal is computed for lags 0 and 1.
7. The velocity estimate is obtained using equation 27.

For fast moving blood flow, where the velocity can be as high as 1 m/s, the velocity is not constant within a pulse repetition interval. As such, equation 18 may be utilized. However, the depth of the blood vessel can be assumed to be constant, where

$${IF}_{up} ( t ) = Acos ( 2 π ( \frac{2 Bd}{{cT}_{s}} t + \frac{2 {f}_{0}}{c} {\int }_{0}^{t} v ( τ ) d τ + \frac{2 B}{{cT}_{s}} {\int }_{0}^{t} τv ( τ ) d τ ) ) . \qquad ( 28 )$$

The instantaneous frequency of the demodulated up chirp and down chirp signal is therefore

$${f}_{up} ( t ) = \frac{2 Bd}{{cT}_{s}} + \frac{2 {f}_{0}}{c} v ( t ) + \frac{2 B}{{cT}_{s}} tv ( t ) ; \qquad ( 29 )$$

$${f}_{down} ( t ) = - \frac{2 Bd}{{cT}_{s}} + \frac{2 {f}_{e}}{c} v ( t ) - \frac{2 B}{{cT}_{s}} tv ( t ) .$$

The instantaneous frequency can be estimated using spectral (e.g., frequency domain) methods, as well as autocorrelation (e.g., time domain) methods. The process for analyzing fast-moving blood flow imposes tradeoffs between range and velocity resolution that can be improved by borrowing inspiration from nature. As an example, bats utilize chirp signals for echolocation, but they do not use linear frequency modulated chirps. Rather, they adjust the sweep rate to optimize range and velocity resolution. A similar principle can be applied, where

$$IF ( t ) = Acos ( 2 π ( {\int }_{0}^{t} [ B [ {f}_{m} ( t ) - {f}_{m} ( t - \frac{2 d}{c} ) ] + \frac{2 v}{c} [ {f}_{0} + {Bf}_{m} ( t - \frac{2 d}{c} ) ] ] d τ ) ) . \qquad ( 30 )$$

As an example, the process for analyzing fast-moving blood flow may be a down chirp with

$${f}_{m} ( t ) = \frac{1}{{T}_{s}} {e}^{- t} .$$

As such, the intermediate frequency signal simplifies

$$IF ( t ) = Acos ( 2 π ( {\int }_{0}^{t} \frac{B}{{T}_{s}} [ 1 - ( 1 - \frac{2 v ( τ ) B}{{cT}_{s}} ) {e}^{\frac{2 d ( τ )}{c}} ] {e}^{- τ} d τ + \frac{2 {f}_{0}}{c} {\int }_{0}^{t} v ( τ ) d τ ) ) . \qquad ( 31 )$$

The parameters B and Tₛ may be chosen such that the Doppler frequency shifts and range frequency shifts are significantly separated. The analysis can be performed by selecting different time windows within the period of the modulation function to isolate Doppler shift or range shift.

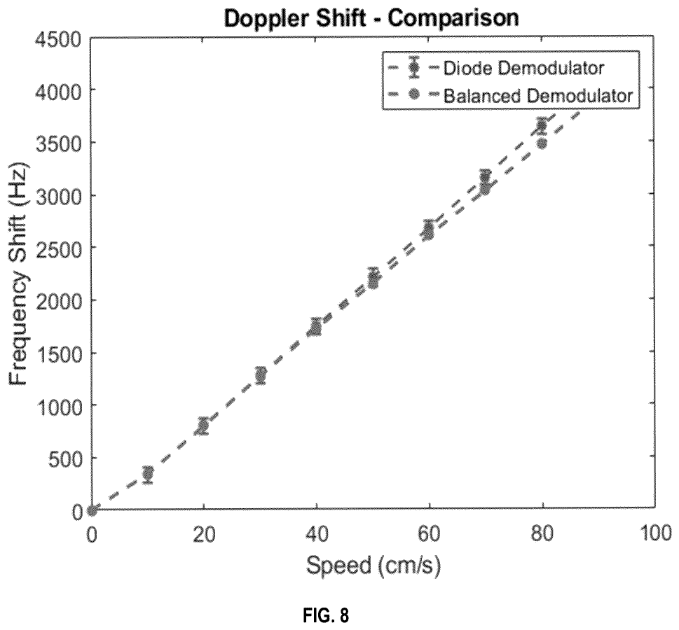

In an example, the mixed signal may be demodulated based on one or more methods, such as a diode-based demodulation process or mixer-based demodulation process. As an example, in a diode demodulator, RF transmit and receive signals may be combined in a non-linear element (e.g., a diode detector), which may produce a difference frequency (e.g., an envelope detector that can extract the beat frequencies). As an example, the diode-based demodulation process may not require a separate local oscillator or high-speed mixer chip. However, the diode-based demodulation process can suffer from lower sensitivity for very weak echoes and may provide only a magnitude of a beat signal. The mixer-based demodulation process uses an active analog mixer (or multiplier) with the transmitted chirp as one input and the received signal as the other. This allows some phase information to be preserved when utilizing the I-Q (e.g., in-phase and quadrature phase) demodulation schemes. As shown in FIG. 8, Doppler shifts using the diode-based demodulation process versus a balanced demodulation process may be compared.

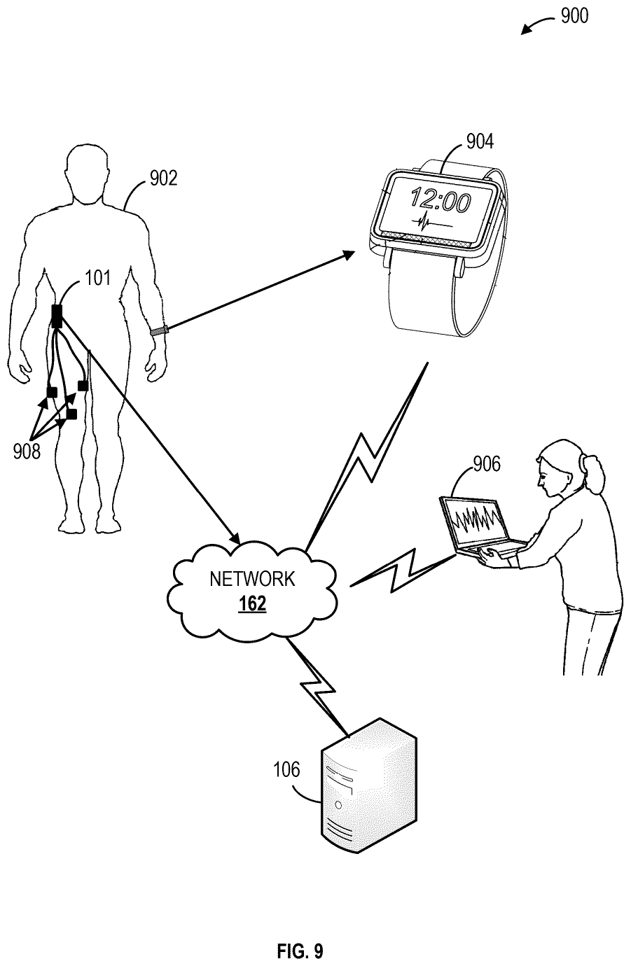

FIG. 9 shows an example system environment 900 for measuring tissue depth and kinematics. The system environment 900 may comprise a computing device 101 in communication with a first user device 904, a second user device 906, and one or more servers 106 via a network 162. The computing device 101 may comprise a mobile device, a smart phone, a tablet computer, and the like. The computing device 101 may include one or more single transducer elements 908 positioned on one or more locations of an individual 902. In an example, the one or more locations may be associated with a muscle and/or tendon region of the individual 902. For example, the one or more locations may be associated with one or more tissues of the of the individual 902. The network 162 may comprise a wireless communication network such as a telecommunications network, a computer network (e.g., LAN or WAN), the Internet, a telephone network, and the like. In an example, the computing device 101 may be in communication with one or more of the first user device 904, the second user device 906, and/or the one or more servers 106 via near-distance communications such as Wireless Fidelity (WiFi), Bluetooth, Near Field Communication (NFC), Global Navigation Satellite System (GNSS), and the like.

The computing device 101 may be configured to generate and transmit one or more ultrasound signals via the one or more single transducer elements 908. The computing device 101 may receive one or more backscattered ultrasound signals based on transmitting the one or more ultrasound signals. For example, the one or more ultrasound signals may backscatter as the one or more ultrasound signals interact with an interior medium of the individual 902, wherein the backscattered ultrasound signals may be received by the computing device 101. The computing device 101 may generate a mixed signal based on the one or more ultrasound signals and the one or more backscattered ultrasound signals. For example, the computing device 101 may generate the mixed signal based on mixing the one or more ultrasound signals with the one or more backscattered ultrasound signals. The computing device 101 may generate an intermediate frequency signal based on applying a lowpass filter to the mixed signal. The computing device 101 may extract range information associated with tissue depth associated with the one or more locations and kinematic (e.g., Doppler) information associated with tissue velocity associated with the one or more locations. As an example, the computing device 101 may generate one or more physiological measurements associated with one or more areas of the individual based on the range information associated with the one or more locations and the kinematic information associated with the one or more locations. The one or more physiological measurements may comprise one or more of muscle and displacement, muscle contraction velocities, tendon velocities, muscle and tendon strain, blood flow velocities, artery wall velocities and displacement, artery wall blood pressure pulse wave velocity, lung motion, cardiac kinematics and blood flow, brain tissue motion and blood flow, or other tissue kinematics. The computing device 101 may be configured to output the one or more physiological measurements to one or more of the first user device 904, the second user device 906, and/or the one or more servers 106, wherein one or more of the first user device 904, the second user device 906, and/or the one or more servers 106 may display information associated with the one or more physiological measurements. For example, the first user device 904 may comprise a smartwatch that displays the information associated with the one or more physiological measurements received from the computing device 101. For example, the second user device 906 may comprise a laptop computer, a desktop computer, a tablet computer, a mobile device, a smart phone, and the like, that displays the information associated with the one or more physiological measurements received from the computing device 101. In an example, the second user device 906 may be configured with one or more transducer elements for transmitting ultrasound signals and determining the range information associated with the tissue depth and the kinematic information associated with the tissue velocity.

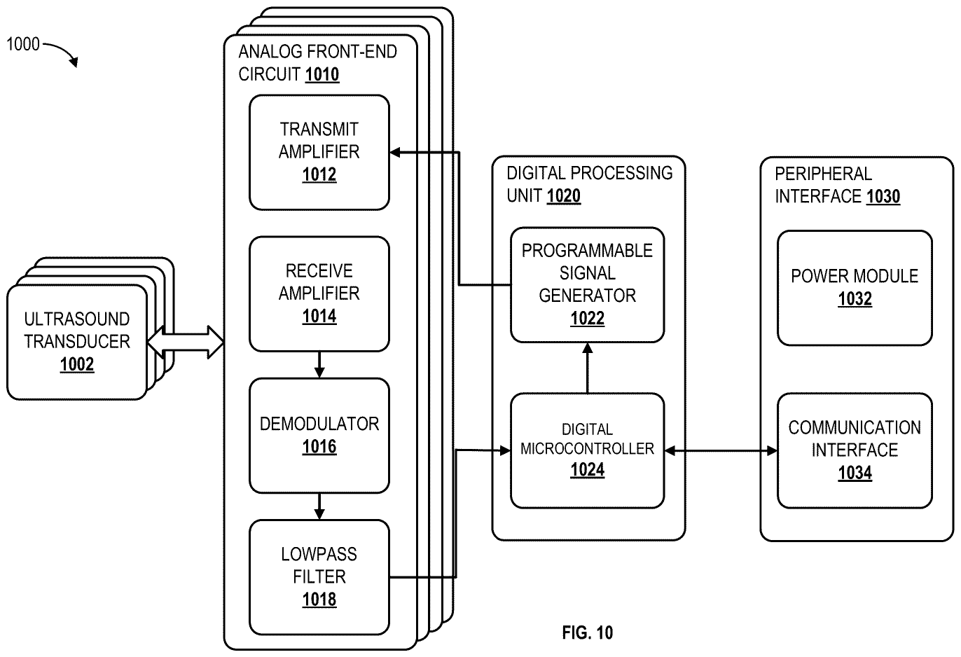

FIG. 10 shows example time delay spectrometry Doppler system architecture 1000. The time delay spectrometry Doppler system architecture 1000 may comprise one or more ultrasound transducers 1002 in communication with one or more analog front-end circuits 1010, a digital processing unit 1020, and a peripheral interface 1030. The ultrasound transducer may comprise a broadband piezoelectric element designed to operate in the MHz range. The one or more analog front-end circuits 1010 may each comprise a transmit amplifier 1012, a receive amplifier 1014, a demodulator 1016, and a lowpass filter 1018. The transmit amplifiers 1012 may receive a signal from the digital processing unit 1020. For example, the digital processing unit 1020 may comprise a programmable signal generator 1022 and a digital microcontroller 1024.

The programmable signal generator 1022 may generate and output a linear chirp signal that may be received by the transmit amplifier 1012. As an example, the programmable signal generator 1022 may be implemented using a direct digital synthesis (DDS) chip controlled by the digital microcontroller 1024. For example, the programmable signal generator 1022 may generate a periodic frequency modulated transmission signal at a designed pulse repetition frequency (PRF) (e.g., 100 Hz) for transmitting frequency modulated chirp signals simultaneously via the one or more ultrasound transducers 1002. In one example, a frequency sweeper may produce a linear FM sweep from frequency f₀ to fₑ over Tₛ. In another example, a programmable chirp generator, or a frequency modulation circuit, may generate the linear chirp signal. The transmit amplifiers 1012 may amplify the linear chirp signal received from the programmable signal generator 1022 and send amplified linear chirp signals to the ultrasound transducers 1002. As an example, a 5 V peak-to-peak signal may be utilized. As an example, adequate signal-to-noise may be maintained even for low voltage transmissions because the continuous wave and correlation processing provide significant gain (e.g., processing gain), allowing detection of echoes that would be too weak in a single short pulse.

The ultrasound transducers 1002 may then output the amplified linear chirp signals received from the transmit amplifiers 1012. The one or more ultrasound transducers 1002 may receive one or more backscattered ultrasound signals based on the transmission of the amplified linear chirp signals. For example, the one or more ultrasound signals may backscatter as the one or more ultrasound signals interact with an interior medium of the individual, wherein the backscattered ultrasound signals may be received by the ultrasound transducers 1002. The ultrasound transducers 1002 may output the backscattered ultrasound signals to the analog front-end circuits 1010. For example, one or more of the receive amplifiers 1014 may receive and amplify one or more of the backscattered ultrasound signals. One or more of the receive amplifiers 1014 may then output the amplified backscattered ultrasound signals to the demodulator 1016. The demodulator 1016 may extract one or more beat frequencies (e.g., difference between transmit and receive) from the amplified backscattered ultrasound signals received from the receive amplifiers 1014 generating an intermediate frequency signal. In an example, a diode-based demodulation process or a mixer-based demodulation process may be implemented. With regard to the diode-based demodulation process, the linear chirp signals and the backscattered ultrasound signals may be combined in a nonlinear element (e.g., a diode detector), which may produce a difference frequency (e.g., an envelope detector that can extract the beat frequencies). As an example, the diode-based demodulation process may not require a separate local oscillator or high-speed mixer chip. However, the diode-based demodulation process may suffer from lower sensitivity for very weak echoes and may provide only a magnitude of a beat signal. The mixer-based demodulation process may utilize an active analog mixer (or multiplier) with the transmitted linear chirp signal as one input and the received signal as the other. This allows some phase information to be preserved when utilizing the I-Q (e.g., in-phase and quadrature phase) demodulation schemes.

The demodulator 1016 may output the intermediate frequency signal to be filtered by the lowpass filter 1018. The filtered intermediate frequency signal may be output to the digital microcontroller 1024. The digital microcontroller 1024 may digitize the intermediate frequency signal with a sampling frequency using an onboard analog-to-digital converter of a microprocessor (e.g., 60-100 kSa/s per channel, at 16 bits/sample) of the digital microcontroller 1024. In an example, the digital microcontroller 1024 may execute firmware that controls the programmable signal generator 1022. In addition, the digital microcontroller 1024 may perform digital signal analysis and may interface with the peripheral interface 1030. For example, the peripheral interface 1030 may comprise a power module 1032 and a communication interface 1034. The digital microcontroller 1024 may interface with the communication interface 1034 of the peripheral interface 1030. As an example, increasing the sweep rate by decreasing the chirp duration may increase the range of detectable Doppler velocities without aliasing. In an example, the digitized intermediate frequency signal may be processed locally on the microprocessor or transmitted via the communication interface 1034 to a central processing unit. In an example, the digital microcontroller 1024 may analyze the intermediate frequency signal to estimate range-resolved velocities of moving tissue and blood and may transfer results of the analysis from its memory storage to the peripheral interface 1032. The digital microcontroller 1024 may transmit the results to one or more external devices (e.g., an external prosthetic control system, an exoskeleton control system, or a neuromuscular electrical stimulation system). As an example, the digitized signals may be stored in the memory storage of the digital microcontroller 1024 and transferred to the communication interface 1034, wherein the communication interface 1034 may then transmit the results to a central processing unit for further analysis and display to an end user. The power module 1032 may be configured to distribute power to all the components of the time delay spectrometry Doppler system 1000, such as power received from a battery, or from an external USB interface.

As an example, the dual-chirp mode may allow separation and extraction of range and Doppler terms with greater accuracy, leveraging the symmetry of the chirp pair. The analog front-end circuits 1010 may remain highly compact and power-efficient, incorporating low-voltage op-amps for filtering and amplification (e.g., 20 dB gain stages), as well as a transmit/receive switch to protect the receiver path. As an example, analog circuitry of the time delay spectrometry Doppler system 1000 may be deployed on a small, battery-powered PCB, enabling wearable deployment. As an example, the time delay spectrometry Doppler system may run on a rechargeable Li-ion battery.

The time delay spectrometry Doppler system architecture 1000 described in FIG. 10 may represent a technical improvement to several technical problems associated with conventional ultrasound systems. Conventional ultrasound systems typically require high-voltage transmission signals ranging from 20 to 100 volts peak-to-peak and short-duration pulses in the nanosecond to microsecond range, necessitating specialized high-voltage front-end electronics, transmit/receive switches, and high-speed digitization systems operating at 20 to 100 mega-samples per second. These hardware demands create substantial constraints on miniaturization, power consumption, and thermal management. In contrast, the time delay spectrometry Doppler system architecture 1000 may operate using frequency-modulated continuous wave ultrasound signals at voltages as low as 5 volts peak-to-peak with sweep times between 1 millisecond and 100 milliseconds, which may significantly reduce power consumption and simplify hardware requirements. The system architecture 1000 may enable digitization at lower sampling rates (e.g., 60-100 kSa/s per channel) compared to conventional pulse-echo systems, reducing computational complexity and power requirements. Additionally, conventional Doppler ultrasound systems typically lack the ability to simultaneously measure both tissue depth and velocity using a single transducer element, often requiring separate transmit and receive transducers positioned at predetermined angles or complex phased array configurations. The system architecture 1000 may address this limitation by enabling simultaneous extraction of range information and Doppler information from a single transducer element through processing of the intermediate frequency signal generated by the demodulator 1016 and lowpass filter 1018. This approach may eliminate the need for complex transducer arrays and high-voltage switching circuitry, potentially enabling compact, battery-powered implementations suitable for wearable applications. Furthermore, the system architecture 1000 may provide depth-resolved velocity measurements without requiring precise transducer positioning by trained personnel, which may expand accessibility of ultrasound-based monitoring beyond specialized clinical environments to applications such as continuous rehabilitation monitoring, functional assessment during dynamic activities, athletic performance and military fitness assessment, and control of powered prosthetic and orthotic devices, among other applications.

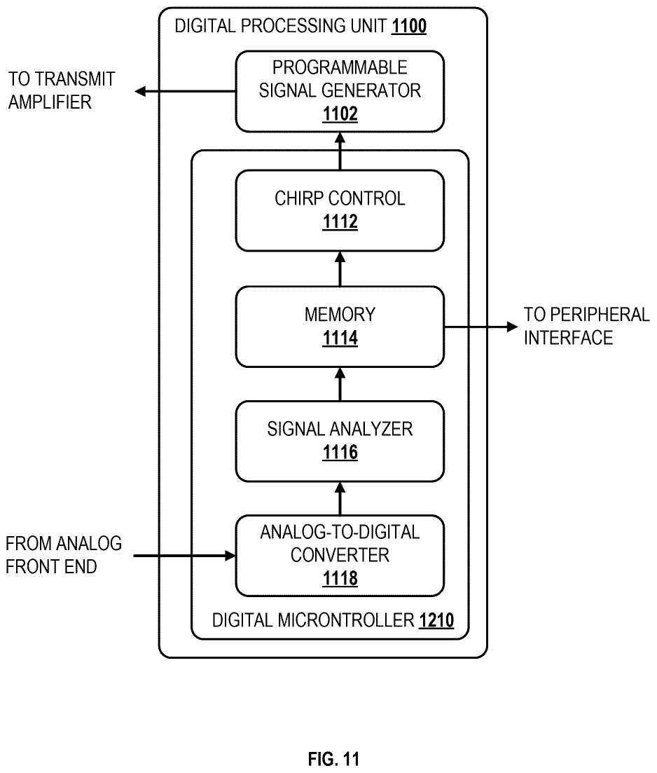

FIG. 11 shows example system architecture of a digital signal processing unit 1100 (e.g., digital signal processing unit 1020). The digital signal processing unit 1100 may comprise a programmable signal generator 1102 (e.g., programmable signal generator 1022) and a digital microcontroller 1110 (e.g., digital microcontroller 1024). The programmable signal generator 1102 may generate and output a linear chirp signal that may be received by a transmit amplifier (e.g., transmit amplifier 1012). As an example, the programmable signal generator 1102 may be implemented using a direct digital synthesis (DDS) chip controlled by the digital microcontroller 1110. The digital microcontroller 1110 may comprise a chirp control module 1112, memory 1114, a signal analyzer module 1116, and an analog-to-digital converter 1118. As an example, the programmable signal generator 1102 may generate a periodic frequency modulated transmission signal at a predetermined pulse repetition frequency (PRF) (e.g., 100 Hz) for transmitting frequency modulated chirp signals simultaneously via one or more ultrasound transducers. In one example, a frequency sweeper may produce a linear FM sweep from frequency f₀ to fₑ over Tₛ. In another example, the chirp control module 1112, or a frequency modulation circuit, may produce the linear chirp signal. The transmit amplifier may amplify the linear chirp signal received from the programmable signal 1102 and send the amplified linear chirp signal to the ultrasound transducers. As an example, a 5 V peak-to-peak signal may be utilized. As an example, adequate signal-to-noise may be maintained even for low voltage transmissions because the continuous wave and correlation processing provide significant gain (e.g., processing gain), allowing detection of echoes that would be too weak in a single short pulse.

The digital microcontroller 1110 may receive a filtered intermediate frequency signal from an analogy front-end circuit (e.g., analog front-end circuit 1010). The analog-to-digital converter 1118 of the digital microcontroller 1110 may digitize the intermediate frequency signal with a sampling frequency. The signal analyzer 1116 of the digital microcontroller 1110 may perform digital signal analysis and send the analyzed signal to the be stored in the memory 1114 of the digital microcontroller 1110. The memory 1114 may interface with a peripheral interface (e.g., peripheral interface 1030). As an example, the signal analyzer 1116 may analyze the intermediate frequency signal to estimate range-resolved velocities of moving tissue and blood. The digital microcontroller 1110 may transmit the results to one or more external devices (e.g., an external prosthetic control system, an exoskeleton control system, or a neuromuscular electrical stimulation system) via the peripheral interface. As an example, the peripheral interface may then transmit the results to a central processing unit for further analysis and display to an end user.

The digital signal processing unit 1100 described in FIG. 11 may represent a technical improvement to several technical problems associated with conventional ultrasound signal processing architectures. Conventional ultrasound systems typically require separate, dedicated hardware components for signal generation, data acquisition, and signal processing, resulting in complex system architectures with multiple interconnected circuit boards and substantial power consumption. These conventional systems often rely on specialized digital signal processors or field-programmable gate arrays that operate at high clock speeds to handle the computational demands of real-time ultrasound signal processing, leading to increased cost, size, and thermal dissipation requirements. In contrast, the digital signal processing unit 1100 may integrate multiple functions within a single microcontroller architecture, combining the programmable signal generator 1102, the analog-to-digital converter 1118, the signal analyzer 1116, and memory 1114 in a unified processing platform. This integration, made possible by the proposed time delay spectrometry method, may reduce system complexity, minimize interconnect delays, and lower overall power consumption compared to distributed processing architectures. The digital signal processing unit 1100 may enable real-time processing of intermediate frequency signals at lower sampling rates compared to conventional pulse-echo systems, reducing the computational burden on the signal analyzer 1116 while maintaining adequate temporal resolution for tissue velocity measurements. Additionally, conventional ultrasound systems often require external computing resources for advanced signal processing algorithms, limiting their suitability for standalone wearable applications. The digital signal processing unit 1100 may address this limitation by performing range-resolved velocity estimation directly on the digital microcontroller 1110, enabling autonomous operation without dependence on external processing resources. The chirp control module 1112 may provide adaptive parameter adjustment capabilities, allowing the system to optimize transmission parameters based on application-specific requirements such as tissue depth, velocity range, and anatomical location. Furthermore, the digital signal processing unit 1100 may facilitate direct interfacing with external control systems through the peripheral interface, enabling low-latency transmission of processed velocity data for applications such as prosthetic control, exoskeleton assistance, and neuromuscular stimulation, which may expand the utility of ultrasound-based sensing beyond traditional diagnostic imaging to real-time human-machine interface applications.

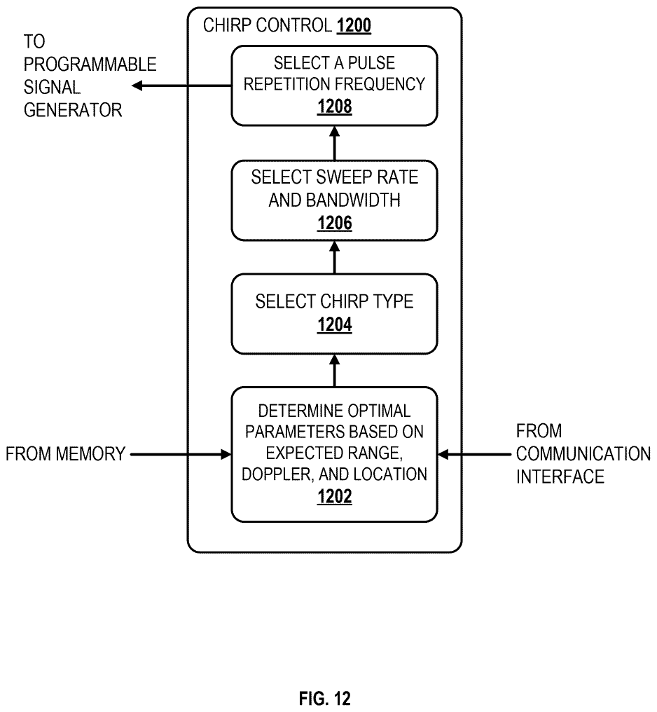

FIG. 12 shows example process that may be implemented by a chirp control module 1200 (e.g., chirp control module 1112). At 1202, the chirp control module 1200 may determine optimal parameters based on expected range, Doppler, and location. In one example, with a linearly-swept chirp, the range frequency shift is given by

$${f}_{range} = \frac{2 B}{{T}_{s}} \frac{d}{c}$$

corresponding to the range of the scatterer d. The sweep time Tₛ can be chosen based on the transducer bandwidth B and the sampling frequency corresponding to the maximum desired range shift ƒ_{range}. In another example, the optimal parameters may be selected from a lookup table in memory based on current estimates of expected range and/or Doppler frequency shifts. In another example, a user may select a parameter set based on a desired performance, such as the ability to detect a maximum Doppler frequency shift without aliasing, the ability to resolve tissue interfaces with a certain desired range resolution, the ability to resolve the speed of moving tissue with a desired Doppler resolution, and/or the ability to achieve a certain signal to noise ratio in the intermediate frequency signal. At 1204, the chirp control module 1200 may select a chirp type. At 1206, the chirp control module 1200 may select a sweep rate and bandwidth. At 1208, the chirp control module 1200 may select a pulse repetition frequency. The chirp control module 1200 may output the parameters to a programmable signal generator (e.g., programmable signal generator 1022, programmable signal generator 1102). In one embodiment, the programmable signal generator may utilize a digital frequency sweep oscillator or a direct digital synthesis (DDS) method, among other possibilities.

The chirp control module 1200 described in FIG. 12 may represent a technical improvement to several technical problems associated with conventional ultrasound parameter optimization. Conventional ultrasound systems typically employ fixed transmission parameters that are predetermined during system design and remain constant across different imaging scenarios, tissue types, and anatomical locations. These fixed parameters often result in suboptimal performance when imaging conditions deviate from the design assumptions, leading to reduced signal quality, limited depth penetration, or inadequate velocity resolution. Conventional systems that do offer parameter adjustment typically require manual intervention by trained operators who must possess specialized knowledge of ultrasound physics and system operation to select appropriate settings. In contrast, the chirp control module 1200 may provide automated parameter optimization based on expected range, Doppler characteristics, and anatomical location, eliminating the need for operator expertise in ultrasound system configuration. The chirp control module 1200 may dynamically adjust chirp type, sweep rate, bandwidth, and pulse repetition frequency to match specific measurement requirements, potentially improving signal-to-noise ratio, range resolution, and velocity accuracy across diverse application scenarios. The ability to select parameters from a lookup table based on current estimates of range and Doppler may enable adaptive operation where the system continuously optimizes its transmission characteristics in response to changing tissue conditions or movement patterns. In another example, the chirp control module can utilize the current estimate of the depth of a moving tissue interface and adaptively adjust a sweep rate to estimate the tissue velocity with high resolution without aliasing. This adaptive capability may be particularly beneficial for wearable applications where transducer placement may vary between users or shift during dynamic activities, as the system can automatically compensate for these variations without requiring manual recalibration. Furthermore, the chirp control module 1200 may allow users to select parameter sets based on desired performance characteristics, providing flexibility to prioritize different measurement objectives such as maximum depth penetration, highest velocity resolution, or lowest power consumption depending on the specific application requirements.

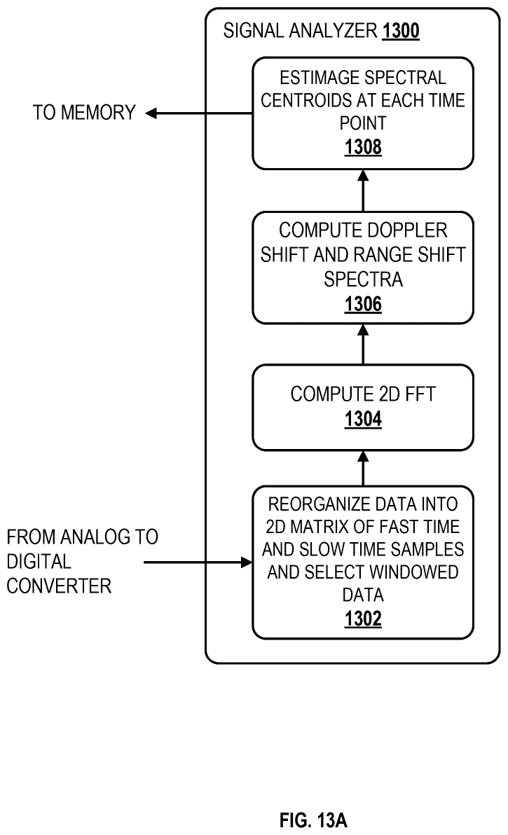

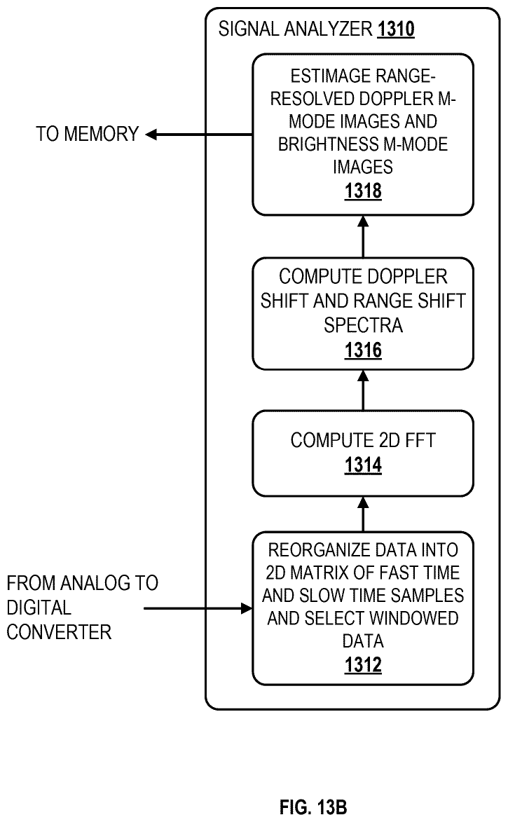

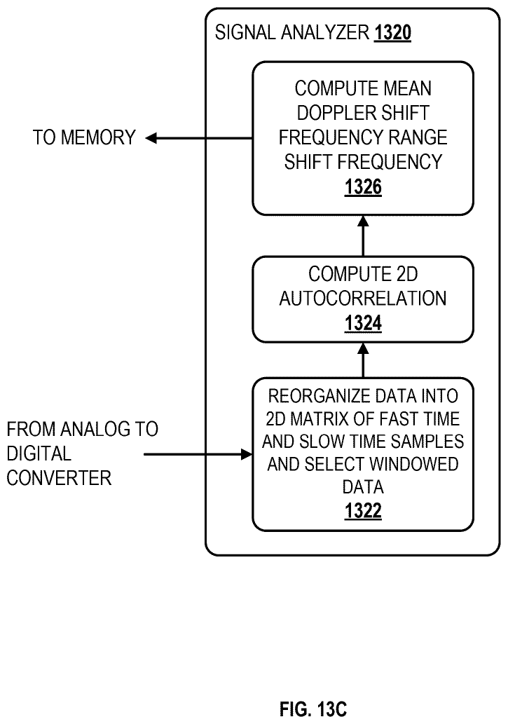

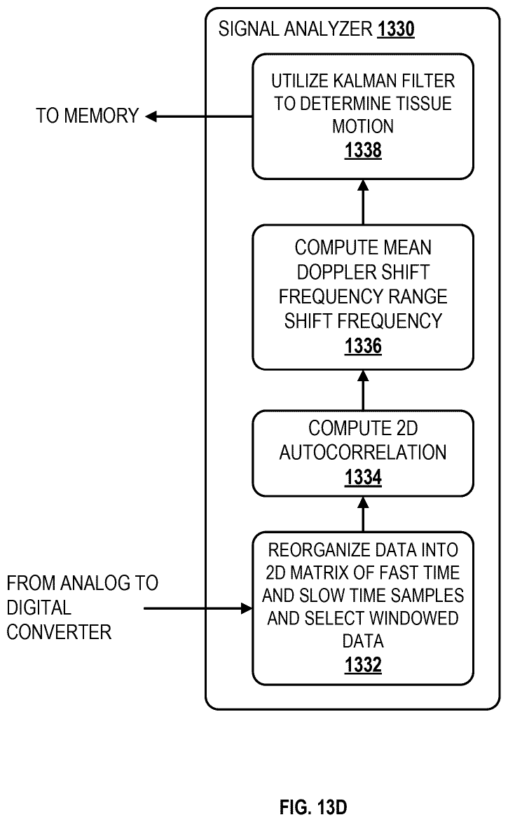

FIGS. 13A-13D show example processes that may be implemented by a signal analyzer (e.g., signal analyzer 1116). As one example, FIG. 13A shows an example process that may be implemented by a signal analyzer 1300 (e.g., signal analyzer 1116). At 1302, the signal analyzer 1300 may receive a digitized signal from an analog-to-digital converter (e.g., analog-to-digital converter 1118), reorganize the digital signal into a 2D matrix of fast time and slow time samples, and select windowed data. At 1304, the signal analyzer 1300 may compute a 2D Fast Fourier Transform (FFT) based on the 2D matrix of the fast time and slow time samples. At 1306, the signal analyzer 1300 may compute Doppler shift and range shift spectra based on the 2D FFT. At 1308, the signal analyzer 1300 may estimate one or more spectral centroids at each time point based on the Doppler shift and range shift spectra and send the estimate of the one or more spectral centroids at each time point to memory storage (e.g., memory 1114).

As another example, FIG. 13B shows an example process that may be implemented by a signal analyzer 1310 (e.g., signal analyzer 1116). At 1312, the signal analyzer 1310 may receive a digitized signal from an analog-to-digital converter (e.g., analog-to-digital converter 1118), reorganize the digital signal into a 2D matrix of fast time and slow time samples, and select windowed data. At 1314, the signal analyzer 1310 may compute a 2D FFT based on the 2D matrix of the fast time and slow time samples. At 1316, the signal analyzer 1310 may compute Doppler shift and range shift spectra based on the 2D FFT. At 1318, the signal analyzer 1310 may estimate range-resolved Doppler M-mode images and brightness M-mode images based on the Doppler shift and range shift spectra and send the estimated range-resolved Doppler M-mode images and the brightness M-mode images to memory storage (e.g., memory 1114).

As another example, FIG. 13C shows an example process that may be implemented by a signal analyzer 1320 (e.g., signal analyzer 1116). At 1322, the signal analyzer 1320 may receive a digitized signal from an analog-to-digital converter (e.g., analog-to-digital converter 1118), reorganize the digital signal into a 2D matrix of fast time and slow time samples, and select windowed data. At 1324, the signal analyzer 1320 may compute a 2D autocorrelation based on the 2D matrix of the fast time and slow time samples. At 1324, the signal analyzer 1320 may compute a mean Doppler shift frequency range shift frequency based on the 2D autocorrelation and send the mean Doppler shift frequency and range shift frequency to memory storage (e.g., memory 1114).

As another example, FIG. 13D shows an example process that may be implemented by a signal analyzer 1330 (e.g., signal analyzer 1116). At 1332, the signal analyzer 1330 may receive a digitized signal from an analog-to-digital converter (e.g., analog-to-digital converter 1118), reorganize the digital signal into a 2D matrix of fast time and slow time samples, and select windowed data. At 1334, the signal analyzer 1330 may compute a 2D autocorrelation based on the 2D matrix of the fast time and slow time samples. At 1336, the signal analyzer 1330 may compute a mean Doppler shift frequency range shift frequency based on the 2D autocorrelation. At 1338, the signal analyzer 1330 may utilize a Kalman filter to determine tissue motion based on the mean Doppler shift frequency range shift frequency and send the tissue motion to memory storage (e.g., memory 1114).

The signal analyzers 1300, 1310, 1320, 1330 described in FIGS. 13A-13D may represent a technical improvement to several technical problems associated with conventional ultrasound signal processing methods. Conventional ultrasound systems typically employ separate processing pipelines for range estimation and velocity measurement, requiring multiple transmission sequences and complex hardware switching between different imaging modes. These conventional approaches often cannot provide simultaneous depth and velocity information from a single acquisition, limiting their utility for real-time monitoring applications where both parameters change dynamically and rapidly. In contrast, the signal analyzers 1300, 1310, 1320, 1330 may extract both range information and kinematic (e.g., Doppler) information from the same intermediate frequency signal through different processing approaches, enabling simultaneous measurement of tissue depth and velocity without requiring mode switching or multiple transmission sequences. The signal analyzer 1300 may provide spectral centroid estimation for applications requiring mean velocity and depth measurements, while the signal analyzer 1310 may generate comprehensive M-mode imaging outputs suitable for clinical visualization and diagnostic assessment. The signal analyzer 1320 may offer computationally efficient autocorrelation-based processing that may reduce power consumption and processing latency compared to frequency-domain methods, which may be beneficial for battery-powered wearable devices. The signal analyzer 1330 may incorporate Kalman filtering to provide enhanced motion tracking with reduced noise sensitivity, potentially improving measurement accuracy in the presence of signal artifacts or low signal-to-noise conditions. The availability of multiple processing approaches may allow the system to select the most appropriate algorithm based on application-specific requirements such as computational resources, power constraints, measurement accuracy needs, or output format preferences. Furthermore, the modular architecture of the signal analyzers 1300, 1310, 1320, 1330 may enable parallel processing implementations where multiple algorithms operate simultaneously on the same data stream, providing redundant measurements for validation or generating different output formats for diverse end-user applications such as control of powered prosthetic and orthotic devices, rehabilitation monitoring, or diagnostic imaging, and other applications.

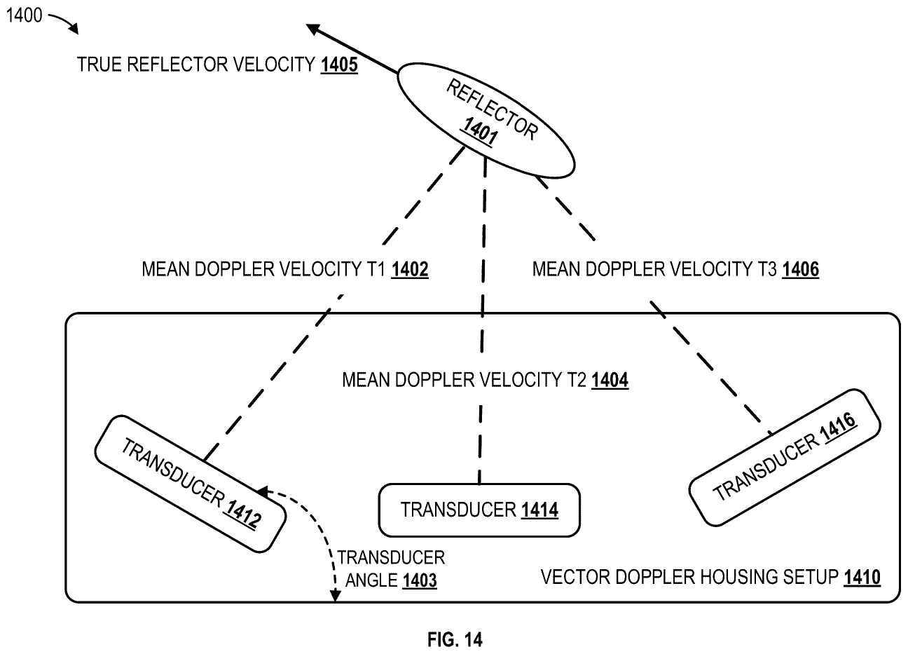

FIG. 14 shows example transducer system architecture 1400. A transducer system may comprise a plurality of transducers 1412, 1414, 1416 housed within a vector Doppler housing setup 1410. The plurality of transducers 1412, 1414, 1416 may be used to estimate a vector of tissue velocity (e.g., magnitude and 3D direction) or blood flow velocity using the principle of vector Doppler. For example, the vector Doppler principle utilizes components of the velocity along an ultrasound beam of each transducer of the plurality of transducers 1412, 1414, 1416 and a known orientation of the transducer, such as a predetermined transducer angle 1403, and estimates the 2D or 3D velocity vector using trigonometric calculations. For example, the transducers 1412, 1414, 1416 may transmit ultrasound signals that may be reflected by reflector 1401. Each signal may be associated with a mean Doppler velocity. For example, a mean Doppler velocity T1 1402 may be estimated for the ultrasound signal transmitted by the transducer 1412, a mean Doppler velocity T2 1404 may be estimated for the ultrasound signal transmitted by the transducer 1414, and a mean Doppler velocity T3 1406 may be estimated for the ultrasound signal transmitted by the transducer 1416. A true reflector velocity 1405 may be determined based on the mean Doppler velocities 1402, 1404, 1406. For example, mean Doppler velocities measured along the three different axes may be combined to yield a true velocity magnitude and direction of movement, overcoming the angle dependence of a single-beam Doppler.

As an example, the transducers 1412, 1414, 1416 may continuously send chirp waveforms and process returning echoes (e.g., backscattered signals) in real time. The output (e.g., the true reflector velocity 1405) may be configured in multiple ways. In one example, a simultaneous Doppler and M-mode display may be configured. For example, the transducer system may produce an M-mode (e.g., motion mode) image by plotting a depth echo intensity (e.g., A-line) as a function of time, while also extracting and overlaying velocity information. As an example, the transducer system may be configured to capture motion of a muscle interface as a bright line on an M-mode image, and at the same time measure its velocity, displaying velocity values or a color map of Doppler shifts. This real-time dual output may be further processed by a central processing unit to extract features and biomarkers, such as tissue strain rate and strain, and also provide a visual display to the user. The transducer system may also be configured to output quantitative metrics (e.g., estimated velocity detected at a certain depth, an integrated velocity over a region, or a Kalman filtered position estimate) which may be used for biofeedback or as a control signal for other external powered devices.

The transducer system architecture 1400 described in FIG. 14 represents a technical improvement to several technical problems associated with conventional single-beam Doppler ultrasound systems. Conventional Doppler ultrasound systems typically measure velocity components only along a single beam direction, resulting in angle-dependent measurements that require precise transducer positioning and knowledge of the angle between the ultrasound beam and the direction of tissue or blood flow movement. This angle dependency introduces measurement errors when the actual flow direction deviates from the assumed angle. Conventional systems often cannot determine the true magnitude and direction of three-dimensional motion without manual repositioning of the transducer or complex phased array configurations. In contrast, the transducer system architecture 1400 may enable angle-independent velocity measurements by utilizing multiple transducers 1412, 1414, 1416 positioned at predetermined transducer angles 1403 to simultaneously measure velocity components along different axes. The system may combine the individual mean Doppler velocities 1402, 1404, 1406 from each transducer through trigonometric calculations to determine the true reflector velocity 1405, representing both the magnitude and three-dimensional direction of tissue or blood flow movement. This multi-axis measurement approach may eliminate the need for precise angular alignment between the transducer and the direction of motion, potentially reducing operator skill requirements and improving measurement accuracy in applications where flow direction varies or is unknown. The vector Doppler housing setup 1410 may provide a compact, integrated configuration that maintains fixed geometric relationships between the transducers 1412, 1414, 1416, enabling consistent vector velocity estimation without requiring manual adjustment or calibration. Furthermore, the ability to determine true velocity vectors may enhance applications such as blood flow volume measurements, where accurate velocity magnitude combined with vessel cross-sectional area enables quantitative flow rate estimation, and complex musculoskeletal motion analysis, where three-dimensional tissue deformation patterns during dynamic activities may be characterized more completely than with single-beam approaches. In another embodiment, the ultrasound beam angle may be altered using a diffraction grating array transducer, which produces beams at different angles depending upon the transmission frequency.

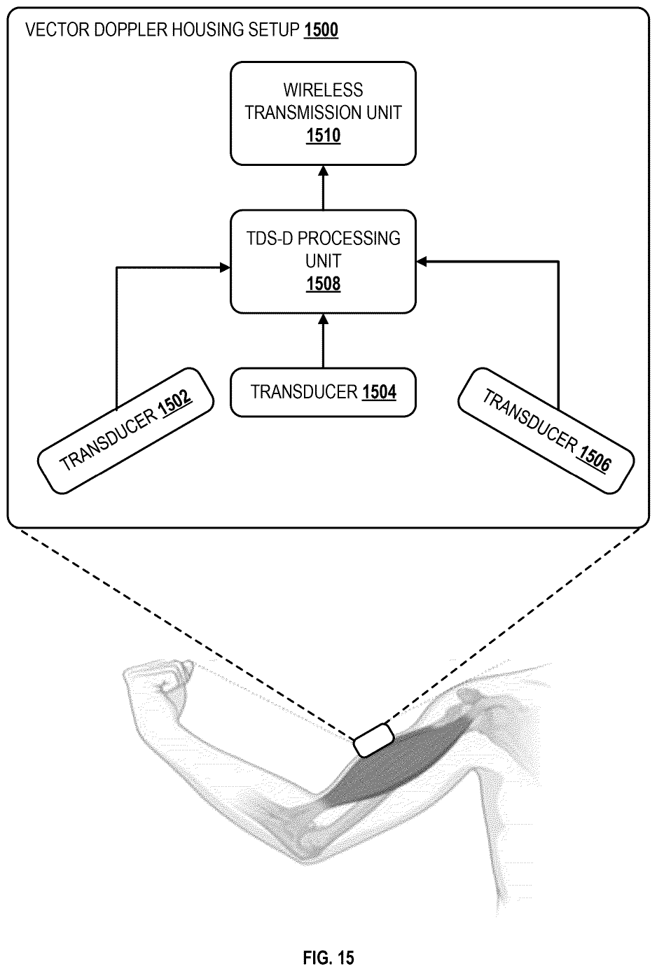

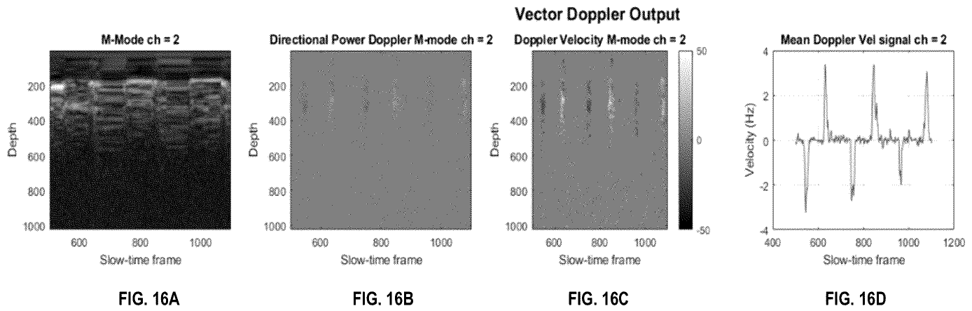

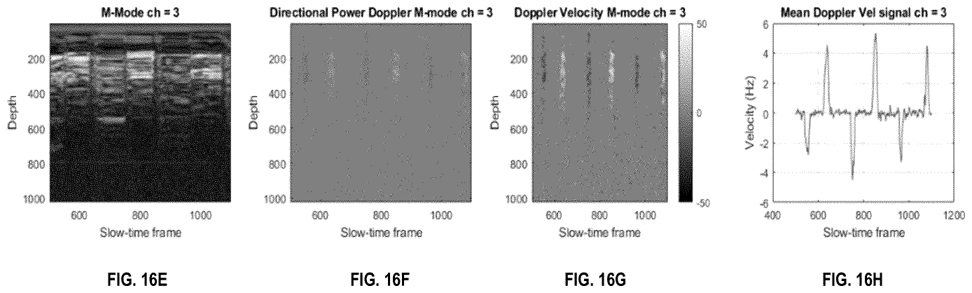

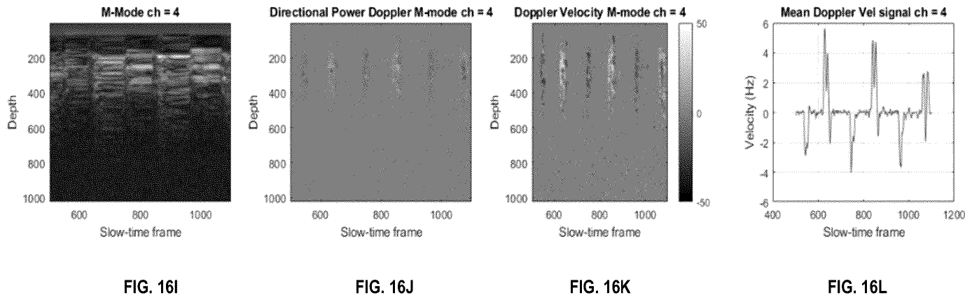

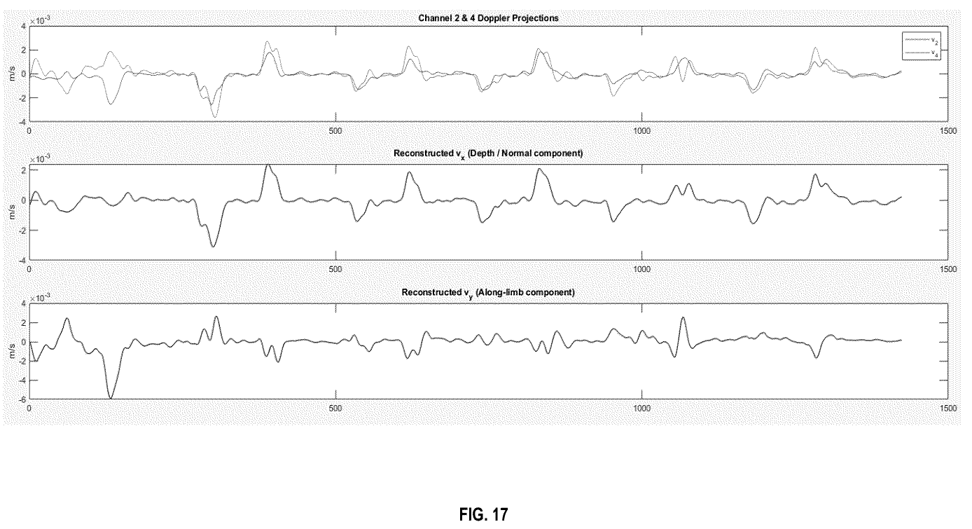

FIG. 15 shows example transducer system architecture 1500. The system architecture 1500 may comprise a vector Doppler housing setup 1500 comprising a plurality of transducers 1502, 1504, 1506, a Time-Delayed Spectroscopy-enabled Doppler (TDS-D) unit 1508, and a wireless transmission unit 1510. The plurality of transducers 1502, 1504, 1506 may comprise a plurality of identical single-channel TDS Doppler units (e.g., transducer elements). As an example, as shown in FIG. 15, three or more transducers 1502, 1504, 1506 may be utilized. For example, vector doppler analysis may be performed by using three or more transducers 1502, 1504, 1506 placed at pre-determined angles with each other on a location of an individual. For example, TDS-D unit 1508 may be configured to measure velocity components along three different axes, which can be combined to yield a true velocity magnitude and direction of movement, overcoming the angle dependence of a single-beam Doppler. For example, to accurately measure blood flow in an artery or muscle fiber shortening velocity of a multi-pennate muscle with varying pennation angles, the three or more transducers 1502, 1504, 1506 may be placed such that beams of each of the transducers 1502, 1504, 1506 intersect a vessel (e.g., a position/location on an individual) at different known angles. Velocity readings from the transducers 1502, 1504, 1506 (each along its beam) may be resolved into an actual flow vector, providing angle-independent flow speed. In an example, the placement approach of the transducers 1502, 1504, 1506 may also map 3D motion of muscles or tendons, providing a more complete picture of muscle deformation during dynamic movement. As such, the transducer system with range resolved Doppler estimation using single transducers may be implemented in a wearable scenario (e.g., a small patch around a limb). Thus, complex musculoskeletal motions may be analyzed, or blood flow volume may be measured based on monitoring vascular systems of an individual (e.g., combining cross-sectional area with flow velocity vector). As shown in FIGS. 16A-16L muscle velocity may be extracted from three channels (e.g., the three or more transducers 1502, 1504, 1506) of a vector Doppler setup. For example, FIGS. 16A-16D show data indicative of muscle velocity extracted from a first transducer of the three or more transducers 1502, 1504, 1506, FIGS. 16E-16H show data indicative of muscle velocity extracted from a second transducer of the three or more transducers 1502, 1504, 1506, and FIGS. 16I-16L show data indicative of muscle velocity extracted from a third transducer of the three or more transducers 1502, 1504, 1506. As an example, as shown in FIG. 17, movement velocity vectors may be resolved using two orthogonal transducers.

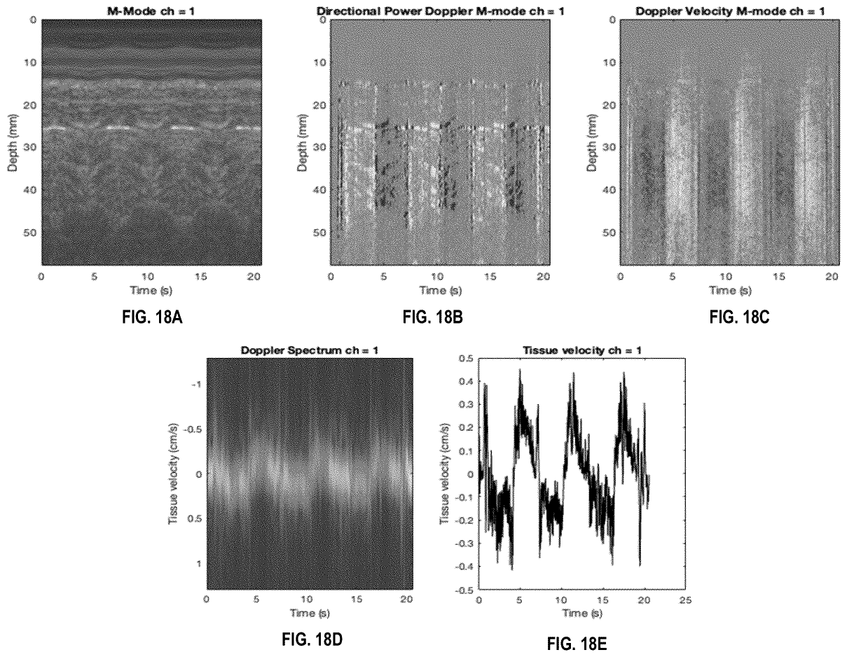

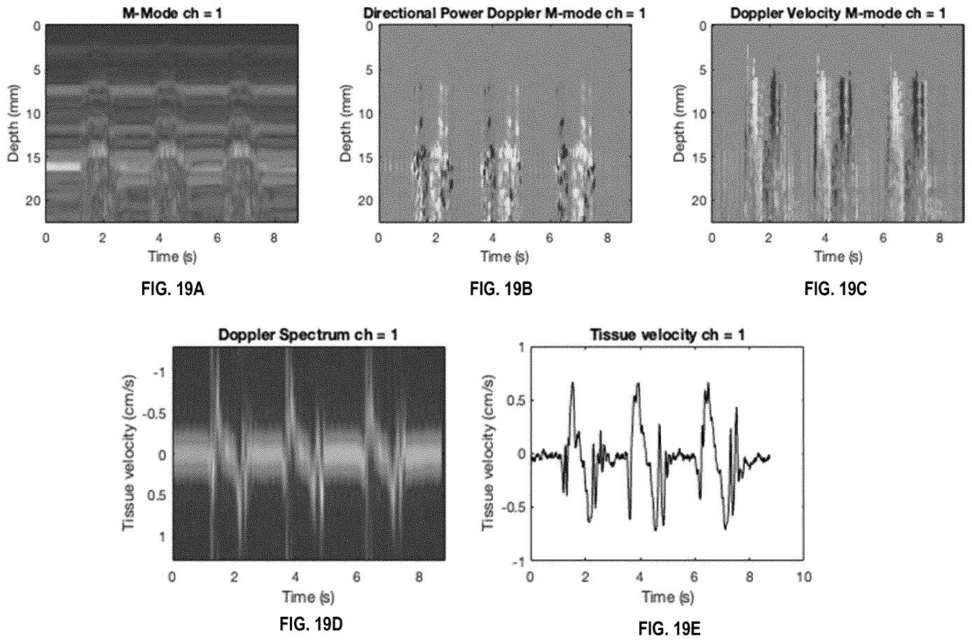

As an example, the transducer system may be used for physical therapy and rehabilitation monitoring. For example, one of the transducers 1502, 1504, 1506 may be placed over a target muscle (e.g., quadriceps, hamstrings, biceps, calves, etc.) to continuously monitor velocity data/information of muscle contractions during exercise. As an example, the velocity data/information may be used to ensure patients/individuals are performing movements at a correct speed to build strength without risk of injury. In an example, the transducer system may be configured to quantify improvement. For example, as a patient recovers strength, the patient may be able to contract a muscle faster or more powerfully (e.g., reflected in higher peak Doppler velocities). By tracking the velocity data/information over the course of therapy sessions and weeks of rehab, clinicians may determine an objective measure of muscle function improvement or neuromuscular control. Tracking muscle contraction velocity may inform force-velocity curves which may be utilized to validate muscle health metrics. Additionally, velocity feedback may be provided to the patient in real time. For example, the velocity information may be used to determine and display biofeedback data or cues (e.g., green light when within desired speed, red if too fast/slow). The feedback may be provided in order to help patients engage the correct muscle groups. As an example, depth information may be provided which may be utilized to confirm the transducer is over the muscle of interest and not, on a bone or unrelated structure. As shown in FIGS. 18A-18E, muscle interface displacement and muscle shortening velocity of the vastus lateralis muscle may be estimated during continuous passive movement using an isokinetic dynamometer. As shown in FIGS. 19A-19E, muscle interface displacement and muscle shortening velocity of the vastus lateralis muscle may be estimated during a body weight squat.

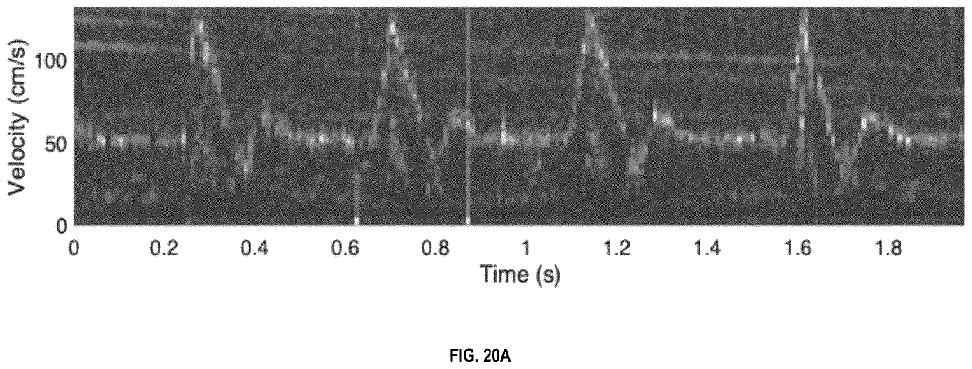

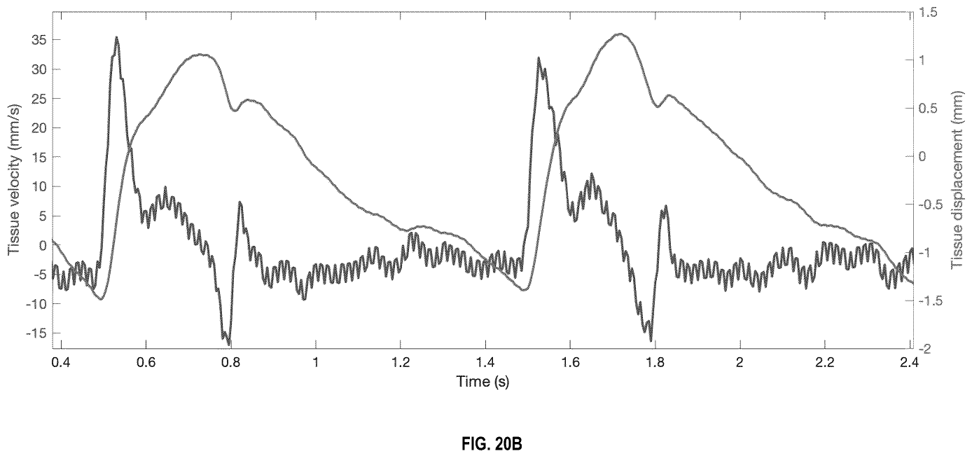

As an example, the transducer system may be used for continuous hemodynamic monitoring. The transducers 1502, 1504, 1506 may be applied to superficial arteries (e.g., radial artery in the wrist, carotid artery in the neck, or femoral artery in the leg) to monitor blood flow velocity continuously. For example, vascular stenosis or occlusion may be monitored in patients with peripheral artery disease or post-surgery. In addition, cardiac output and blood pressure may be continuously monitored. For example, after a surgical bypass or stent placement, a wearable Doppler patch may be placed over an artery in order to monitor changes in flow that might indicate re-narrowing (e.g., restenosis) or thrombosis. In another example, a wearable Doppler patch may be placed on a patient's neck in order to measure carotid flow or may be placed on the femoral artery in order to provide continuous vital information regarding circulation, including measures of cardiac output and blood pressure. In an example, the flow velocity may be integrated with vessel area in order to estimate blood flow volume. In an example, trends in the Doppler waveform shape (e.g. a damped waveform could suggest upstream blockage) may be tracked. In an example, the transducer system may be configured to provide continuous hemodynamic monitoring in neonatal applications. In an example, the transducer system may be configured to provide peripheral blood flow measurements, which may be used for endurance training feedback or detecting dehydration (e.g., changes in peripheral circulation). As shown in FIG. 20A, blood velocity may be tracked over time. As shown in FIG. 20B, the arterial wall velocity and displacement may be tracked over time and used to noninvasively and continuously estimate blood pressure in real time.

As an example, the transducer system may be used for generating B-mode images. For example, the swept frequency transmit signal may be applied to a diffraction grating transducer, where each alternate element may be driven 1800 out of phase. The main lobe may then be suppressed and the ultrasound energy may be transmitted along a grating lobe that depends on the transmission frequency. As such, the ultrasound beam may be steered and B-mode imaging may be implemented, as well as vector Doppler imaging.

The transducer system architecture 1500 described in FIG. 15 represents a technical improvement to several technical problems associated with conventional wearable ultrasound monitoring systems. Conventional wearable ultrasound devices typically employ single-axis velocity measurements that provide limited information about complex three-dimensional tissue motion patterns, and conventional systems often require separate hardware configurations for different measurement modalities such as M-mode imaging, Doppler velocity estimation, and vector flow mapping. These limitations may restrict the utility of wearable ultrasound systems for applications requiring comprehensive motion analysis during dynamic activities. In contrast, the transducer system architecture 1500 may integrate multiple transducers 1502, 1504, 1506 with a TDS-D unit 1508 and wireless transmission unit 1510 in a compact, unified configuration that enables simultaneous multi-axis velocity measurements and depth-resolved tissue motion tracking. The plurality of transducers 1502, 1504, 1506 positioned at predetermined angles, or using diffraction grating arrays that produce angled beams, may provide velocity components along different axes that can be combined to determine true three-dimensional velocity vectors, while the TDS-D unit 1508 may process signals from each transducer to extract both range and kinematic (e.g., Doppler) information simultaneously. This integrated approach may eliminate the need for separate hardware systems or sequential measurement modes, potentially reducing system complexity and enabling continuous monitoring of complex musculoskeletal motions during rehabilitation exercises or athletic activities. The wireless transmission unit 1510 may facilitate untethered operation and real-time data transmission to external processing or display devices, addressing mobility constraints associated with conventional tethered ultrasound systems. Furthermore, the compact form factor of the vector Doppler housing setup 1500 may enable implementation as a wearable patch that can be positioned around a limb or over target anatomical regions, providing continuous monitoring capabilities without restricting movement or requiring constant supervision by trained operators. The ability to map three-dimensional muscle deformation patterns and measure angle-independent blood flow velocities in a single wearable device may expand the applicability of ultrasound-based monitoring to applications such as home-based rehabilitation, athletic performance optimization, and continuous vascular health assessment that were previously impractical with conventional ultrasound systems.

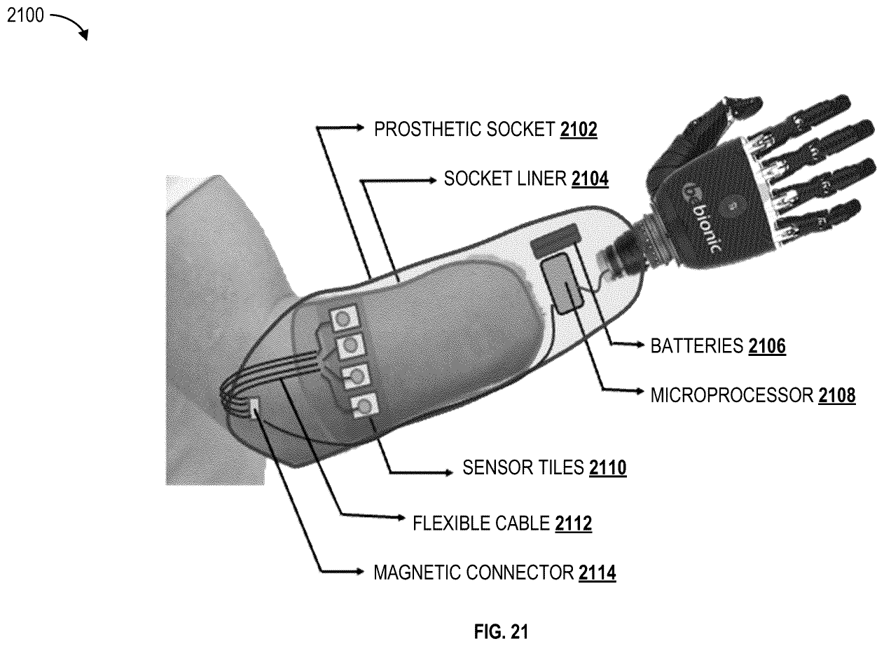

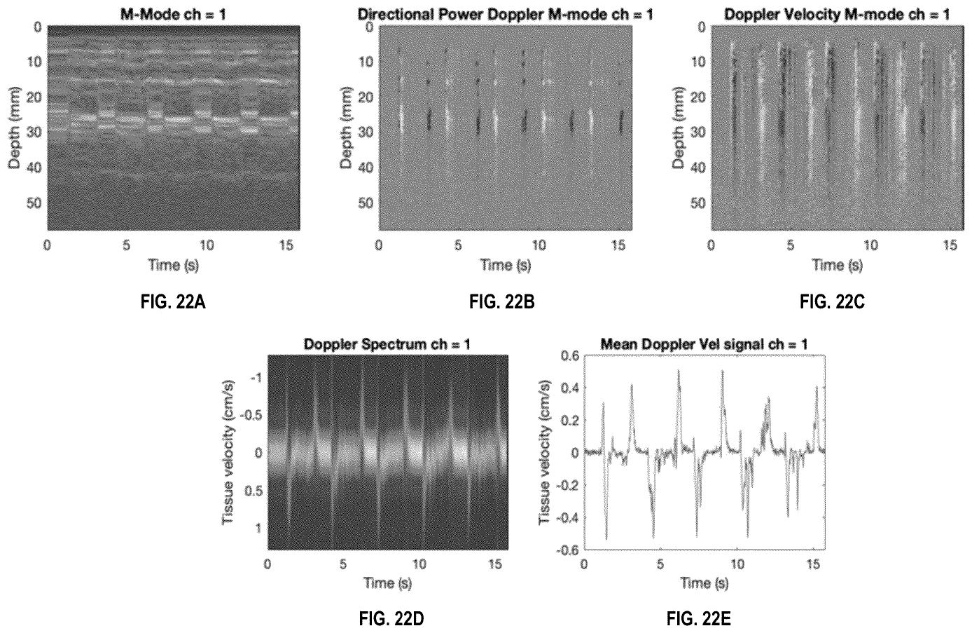

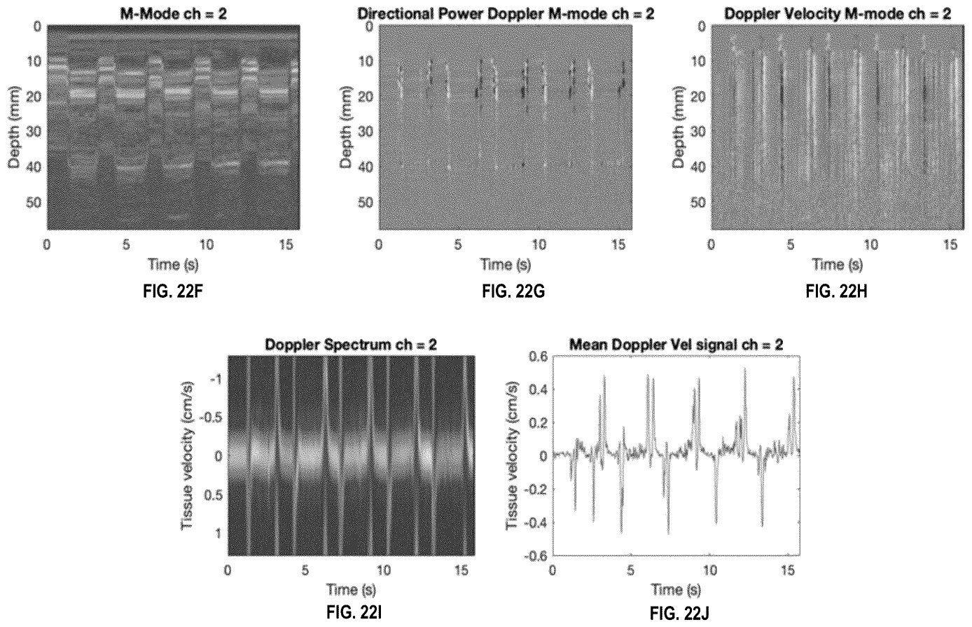

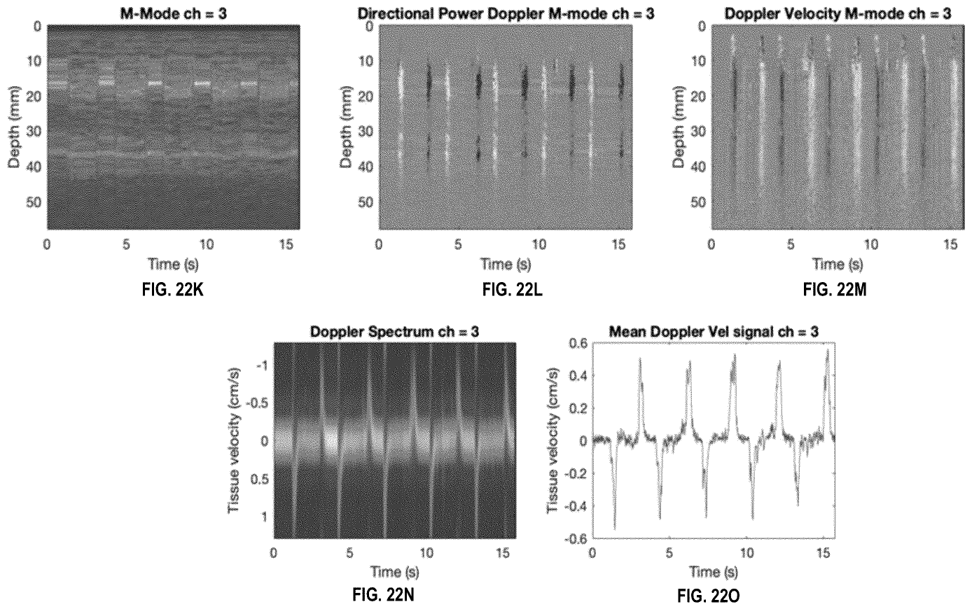

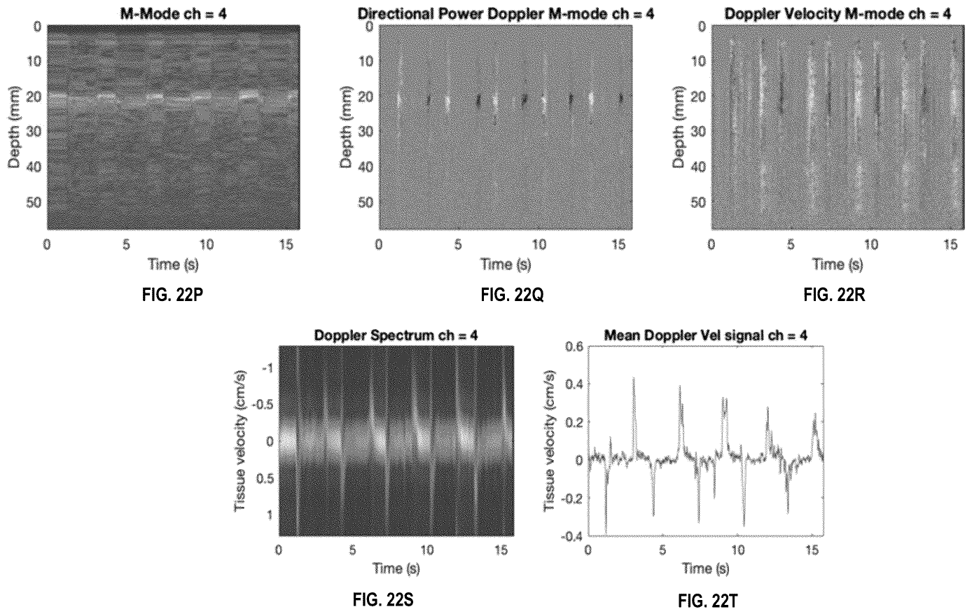

FIG. 21 shows an example prosthetic 2100 that may be controlled based on a range information and kinematic (e.g., Doppler) information received from a transducer system. For example, the prosthetic 2100 may comprise a prosthetic socket 2102, a socket liner 2104, one or more batteries 2106, a microprocessor 2108, one or more sensor tiles 2110, a flexible cable 2112, and a magnetic connector 2114. In an example, the range information and kinematic information output by the transducer system may be utilized to control an exoskeleton. As an example, ultrasound signals may be utilized in order to directly measure the mechanical activation of muscles. The transducer system may be embedded, via the sensor tiles 2110, in the prosthetic socket 2102 of the prosthetic 2100 to monitor a motion of residual muscle groups in a remaining portion of an individual's limb (e.g., residual limb). When the individual attempts to move, the muscles in the residual limb will contract and produce motion. Transducers of the transducer system, via the transducer tiles 2110, may detect a velocity of that movement internally. Data indicative of the velocity may be utilized as a control signal to drive joints of the prosthetic 2100. Because the Doppler is depth-resolved, a transducer may be positioned on the individual to focus on a deep muscle. In an example, for exoskeletons (e.g., wearable robots that assist movement), transducers positioned on key muscles (e.g., quadriceps, hamstrings, biceps, calves, etc.) may feed into the control system to determine the individual's intended movement speed or force. For example, if the quadriceps start contracting rapidly (e.g., high velocity), it may indicate an intent to stand up from a chair, wherein the information output by the transducers may control the exoskeleton to assist the individual in standing up. In another example, ultrasound imaging may be used to decode finger movements for robotic hand control. As an example, as shown in FIGS. 22A-22T, range resolved Doppler images may be obtained using four separate transducers from a residual forearm of an individual with transradial limb loss. For example, FIGS. 22A-22E show range resolved Doppler images associated with a first transducer, FIGS. 22F-22J show range resolved Doppler images associated with a second transducer, FIGS. 22K-220 show range resolved Doppler images associated with a third transducer, and FIGS. 22P-22T show range resolved Doppler images associated with a first transducer.

The prosthetic 2100 described in FIG. 21 represents a technical improvement to several technical problems associated with conventional prosthetic control systems. Conventional prosthetic devices typically rely on surface electromyography (EMG) sensors that measure electrical signals from muscle activity through the skin, which may suffer from signal degradation due to sweat, electrode displacement, skin impedance variations, and cross-talk between adjacent muscle groups. These limitations may result in inconsistent control signals and reduced accuracy in decoding user intent, particularly during dynamic activities or prolonged use. In contrast, the prosthetic 2100 may utilize ultrasound-based sensing through the sensor tiles 2110 embedded in the prosthetic socket 2102 to directly measure mechanical activation of muscles within the residual limb, providing depth-resolved velocity measurements that may be less susceptible to surface conditions and electrode placement variations. The ability to focus on deep muscle groups through depth-resolved Doppler measurements may enable more selective monitoring of specific muscles without interference from superficial tissues, potentially improving the specificity of control signals compared to surface EMG approaches. The sensor tiles 2110 integrated within the prosthetic socket 2102 may maintain consistent positioning relative to the residual limb during normal wear, addressing placement sensitivity issues that may affect external sensor systems. Furthermore, the microprocessor 2108 may process velocity data from the transducer system to generate control signals that directly reflect the speed and force of muscle contractions, providing a more intuitive mapping between user intent and prosthetic movement compared to conventional systems that require complex pattern recognition algorithms to decode EMG signals. The flexible cable 2112 and magnetic connector 2114 may facilitate modular sensor configurations that can be adapted to different residual limb anatomies and prosthetic socket designs, potentially improving the accessibility of advanced prosthetic control across diverse patient populations.

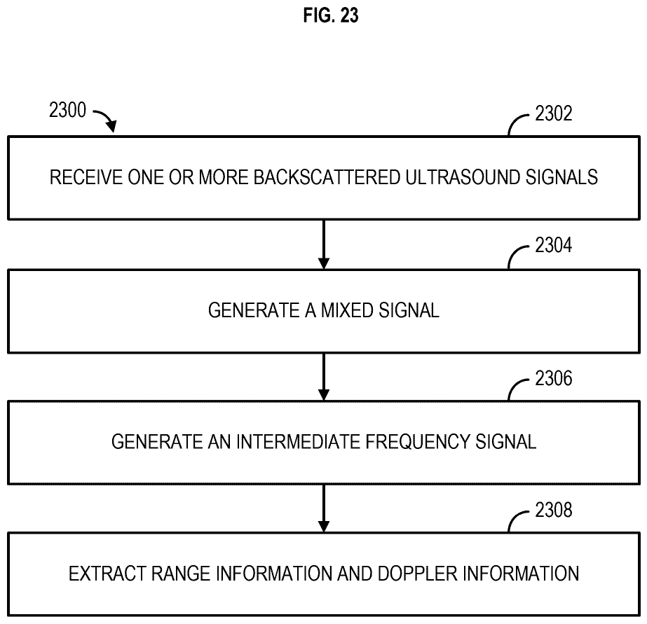

FIG. 23 shows a flowchart of an example method 2300 for measuring tissue depth and velocity using a transducer system. Method 2300 may be implemented by a computing device (e.g., computing device 101, electronic device 102, one or more servers 106, or any combination thereof). At step 2302, one or more backscattered ultrasound signals may be received based on a transmission of one or more ultrasound signals. For example, a computing device (e.g., computing device 101, electronic device 102, one or more servers 106, etc.) may receive the one or more backscattered ultrasound signals via one or more single transducers positioned on one or more locations of an individual based on the transmission of the one or more ultrasound signals. For example, the computing device may transmit the one or more ultrasound signals which may backscatter as the one or more ultrasound signals interact with an interior medium of the individual. The one or more signals may comprise one or more frequency-modulated continuous wave ultrasound signals. The one or more frequency-modulated continuous wave ultrasound signals may operate at a voltage between 0 and 10 volts peak-to-peak and a sweep time may be between 1 millisecond and 100 milliseconds. For example, the one or more ultrasound signals may comprise one or more alternating up-chirp and down-chirp waveforms that sweep between an initial frequency and a final frequency over a sweep time. In an example, the one or more locations may be associated with a muscle and/or tendon region of the individual. For example, the one or more locations may be associated with one or more tissues of the of the individual.

At step 2304, a mixed signal may be generated. For example, the computing device (e.g., computing device 101, electronic device 102, one or more servers 106, etc.) may generate the mixed signal based on the one or more ultrasound signals and the one or more backscattered ultrasound signals. For example, the mixed signal may be generated based on mixing the one or more ultrasound signals with the one or more backscattered ultrasound signals.

At step 2306, an intermediate frequency signal may be generated based on applying a lowpass filter to the mixed signal. For example, the computing device (e.g., computing device 101, electronic device 102, one or more servers 106, etc.) may generate the intermediate frequency signal based on applying the lowpass filter to the mixed signal. For example, the lowpass filter may be used to isolate a difference frequency of the mixed signal and suppress a higher sum frequency resulting in the generation of the intermediate frequency signal. For example, the lowpass filter may be applied to the mixed signal to yield a downmixed intermediate frequency signal that consists of lower frequency components in the audio frequency range representing both range shift frequencies corresponding to scatterers at different depths, as well as Doppler shift frequencies corresponding to the velocity of each scatterer.

At step 2308, range information and kinematic (e.g., Doppler) information may be extracted. For example, the computing device (e.g., computing device 101, electronic device 102, one or more servers 106, etc.) may extract range information associated with tissue depth associated with the one or more locations and kinematic information associated with tissue velocity associated with the one or more locations based on processing the intermediate frequency signal. For example, range information associated with one or more locations of different tissues and kinematic information associated with one or more locations of different tissues may be extracted based on processing the intermediate frequency signal. As one example, the intermediate frequency signal may be processed based on calculating a range frequency based on adding frequency components from the up-chirp and down-chirp waveforms, and calculating a Doppler frequency based on subtracting frequency components from the up-chirp and down-chirp waveforms. As another example, the intermediate frequency signal may be processed based on reorganizing the intermediate frequency signal into a two-dimensional array comprising fast time samples and slow time samples, calculating a range frequency based on analyzing the fast time samples in a time-windowed two-dimensional array, and calculating a Doppler frequency based on analyzing the slow time samples in a time-windowed two-dimensional array comprising. The range information may be determined from the range frequency according to a relationship between bandwidth, the sweep time, and a speed of sound in tissue. The kinematic information may be determined from the Doppler frequency according to a relationship between the initial frequency and the speed of sound in tissue.

In an example, one or more actions may be performed based on extracting the range information and the kinematic information. As one example, one or more physiological measurements associated with one or more areas of the individual may be generated based on the range information associated with the one or more locations and the kinematic information associated with the one or more locations. The one or more physiological measurements may comprise one or more of muscle depth and displacement, muscle contraction velocities, tendon velocities, muscle and tendon strain, blood flow velocities, artery wall velocities and displacement, artery wall blood pressure pulse wave velocity, lung motion, cardiac kinematics and blood flow, brain tissue motion and blood flow, or other tissue kinematics. As another example, one or more medical conditions associated with the individual may be determined based on the range information and the kinematic information. The one or more medical conditions may comprise one or more of one or more cardiovascular conditions, peripheral vascular conditions, cerebrovascular conditions, or musculoskeletal conditions. One or more notifications associated with the one or more medical conditions may be output (e.g., displayed). One or more treatments may be administered to the individual based on the one or more medical conditions. For example, the computing device may be configured with a medical treatment device for administering the one or more treatments to the individual. For example, the computing device may control an external medical treatment device to administer the one or more treatments to the individual. In an example, a response of the individual to a medical treatment or a medical procedure may be determined based on one or more values of the range information and one or more values of the kinematic information, and one or more notifications associated with the response of the individual to the medical treatment or the medical procedure may be output. For example, the one or more values of the range information and the one or more values of the kinematic information may comprise current and historical values of the range information and the kinematic information, respectively. In an example, a measure of functional performance of the individual may be output based on the range information and the kinematic information. In an example, a measure of recovery status of the individual after an injury may be output based on the range information and the kinematic information. As another example, one or more signals for controlling an external device may be output based on the range information and the kinematic information. For example, the external device may comprise a powered prosthetic limb, an exoskeleton, or one or more human machine interfaces. The one or more human machine interfaces may comprise one or more of feedback for neurostimulation, neuromuscular electrical stimulation, or functional electrical stimulation. As another example, image data associated with the one or more locations on the individual may be generated based on the range information and the kinematic information. For example, the computing device may output (e.g., display) the image data. The image data may comprise one or more M-mode ultrasound images indicating locations of tissue interfaces over time or one or more M-mode ultrasound images indicating kinematics of tissue interfaces (including displacement, velocity, acceleration, jerk, strain, and strain rate) over time.

The method 2300 described in FIG. 23 represents a technical improvement to several technical problems associated with conventional ultrasound measurement techniques. Conventional ultrasound systems typically require separate measurement sequences to obtain depth information and velocity information, necessitating mode switching between M-mode imaging for tissue depth tracking and Doppler mode for velocity estimation. These sequential measurement approaches may introduce temporal misalignment between depth and velocity data, limiting their utility for applications requiring synchronized measurements of tissue position and movement during dynamic activities. In contrast, the method 2300 may enable simultaneous extraction of both range information and kinematic information from a single intermediate frequency signal generated from frequency-modulated continuous wave ultrasound transmissions, eliminating the need for mode switching or multiple acquisition sequences. The method 2300 may process the intermediate frequency signal using either frequency-domain approaches that calculate range and Doppler frequencies from up-chirp and down-chirp waveform components, or time-domain approaches that reorganize the signal into two-dimensional arrays of fast time and slow time samples for separate analysis. This dual-parameter extraction capability may provide temporally synchronized depth and velocity measurements that may be particularly beneficial for applications such as control of a powered prosthetic and orthotic devices, rehabilitation monitoring, and cardiovascular assessment where the relationship between tissue position and movement velocity provides information about muscle activation patterns, blood flow dynamics, or joint kinematics. Furthermore, the method 2300 may operate using low-voltage frequency-modulated continuous wave signals rather than high-voltage pulsed transmissions, potentially reducing power consumption and hardware complexity while maintaining adequate signal-to-noise ratio through processing gain achieved by correlation of the transmitted and received chirp waveforms. The ability to generate diverse outputs including physiological measurements, medical condition assessments, device control signals, and imaging data from a single measurement sequence may expand the versatility of ultrasound-based monitoring systems compared to conventional approaches that typically provide only a single type of output per measurement mode.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g., a genetic algorithms), swarm intelligence (e.g., an ant algorithms), and hybrid intelligent systems (e.g., expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, via one or more single transducers positioned on one or more locations of an individual, based on a transmission of one or more ultrasound signals, one or more backscattered ultrasound signals;
generating, based on the one or more ultrasound signals and the one or more backscattered ultrasound signals, a mixed signal;
generating, based on applying a lowpass filter to the mixed signal, an intermediate frequency signal; and
extracting, based on processing the intermediate frequency signal, range information associated with tissue depth associated with the one or more locations and kinematic information associated with range-resolved tissue velocity associated with the one or more locations.

2. The method of claim 1, wherein the one or more ultrasound signals comprise one or more frequency-modulated continuous wave ultrasound signals, wherein the one or more frequency-modulated continuous wave ultrasound signals operate at a voltage between 0 and 10 volts peak-to-peak and a sweep time is between 1 millisecond and 100 milliseconds.

3. The method of claim 1, wherein the one or more ultrasound signals comprise one or more alternating up-chirp and down-chirp waveforms that sweep between an initial frequency and a final frequency over a sweep time.

4. The method of claim 3, wherein processing the intermediate frequency signal comprises:
calculating a range frequency based on adding frequency components from the up-chirp and down-chirp waveforms; and
calculating a Doppler frequency based on subtracting frequency components from the up-chirp and down-chirp waveforms.

5. The method of claim 3, wherein processing the intermediate frequency signal comprises:
reorganizing the intermediate frequency signal into a two-dimensional array comprising fast time samples and slow time samples;
calculating a range frequency based on analyzing the fast time samples in a time-windowed two-dimensional array; and
calculating a Doppler frequency based on analyzing the slow time samples in a time-windowed two-dimensional array.

6. The method of claim 5, wherein the range information is determined from the range frequency according to a relationship between bandwidth, the sweep time, and a speed of sound in tissue, and wherein the kinematic information is determined from the Doppler frequency according to a relationship between the initial frequency and the speed of sound in tissue.

7. The method of claim 1, further comprising:
generating, based on the range information associated with the one or more locations and the kinematic information associated with the one or more locations, one or more physiological measurements associated with one or more areas of the individual, wherein the one or more physiological measurements comprise one or more of muscle depth and displacement, muscle contraction velocities, tendon velocities, muscle and tendon strain, blood flow velocities, artery wall velocities and displacement, artery wall blood pressure pulse wave velocity, lung motion, cardiac kinematics and blood flow, or brain tissue motion and blood flow.

8. The method of claim 1, further comprising:
determining, based on the range information and the kinematic information, one or more medical conditions associated with the individual, wherein the one or more medical conditions comprise one or more of one or more cardiovascular conditions, peripheral vascular conditions, cerebrovascular conditions, or musculoskeletal conditions;
outputting one or more notifications associated with the one or more medical conditions; and
causing, based on the one or more medical conditions, one or more treatments associated with the individual, wherein the one or more treatments comprise one or more of administering medication or administering a medical procedure.

9. The method of claim 1, further comprising:
outputting, based on the range information and the kinematic information, one or more signals for controlling an external device, wherein the external device comprises a powered prosthetic limb, an exoskeleton, or one or more human machine interfaces, wherein the one or more human machine interfaces comprise one or more of feedback for neurostimulation, neuromuscular electrical stimulation, or functional electrical stimulation.

10. The method of claim 1, further comprising:
generating, based on the range information and the kinematic information, image data associated with the one or more locations on the individual, wherein the image data comprises one or more M-mode ultrasound images.

11. An apparatus comprising
one or more processors; and
a memory storing processor-executable instructions that, when executed by the one or more processors, cause the apparatus to:
receive, via one or more single transducers positioned on one or more locations of an individual, based on a transmission of one or more ultrasound signals, one or more backscattered ultrasound signals;
generate, based on the one or more ultrasound signals and the one or more backscattered ultrasound signals, a mixed signal;
generate, based on applying a lowpass filter to the mixed signal, an intermediate frequency signal; and
extract, based on processing the intermediate frequency signal, range information associated with tissue depth associated with the one or more locations and kinematic information associated with range-resolved tissue velocity associated with the one or more locations.

12. The apparatus of claim 11, wherein the one or more ultrasound signals comprise one or more frequency-modulated continuous wave ultrasound signals, wherein the one or more frequency-modulated continuous wave ultrasound signals operate at a voltage between 0 and 10 volts peak-to-peak and a sweep time is between 1 millisecond and 100 milliseconds.

13. The apparatus of claim 11, wherein the one or more ultrasound signals comprise one or more alternating up-chirp and down-chirp waveforms that sweep between an initial frequency and a final frequency over a sweep time.

14. The apparatus of claim 13, wherein the processor-executable instructions, when executed by the one or more processors, further cause the apparatus to:
calculate a range frequency based on adding frequency components from the up-chirp and down-chirp waveforms; and
calculate a Doppler frequency based on subtracting frequency components from the up-chirp and down-chirp waveforms.

15. The apparatus of claim 13, wherein the processor-executable instructions, when executed by the one or more processors, further cause the apparatus to:
reorganize the intermediate frequency signal into a two-dimensional array comprising fast time samples and slow time samples;
calculate a range frequency based on analyzing the fast time samples in a time-windowed two-dimensional array; and
calculate a Doppler frequency based on analyzing the slow time samples in a time-windowed two-dimensional array.

16. The apparatus of claim 15, wherein the range information is determined from the range frequency according to a relationship between bandwidth, the sweep time, and a speed of sound in tissue, wherein the kinematic information is determined from the Doppler frequency according to a relationship between the initial frequency and the speed of sound in tissue.

17. The apparatus of claim 11, wherein the processor-executable instructions, when executed by the one or more processors, further cause the apparatus to:
generate, based on the range information associated with the one or more locations and the kinematic information associated with the one or more locations, one or more physiological measurements associated with one or more areas of the individual, wherein the one or more physiological measurements comprise one or more of muscle depth and displacement, muscle contraction velocities, tendon velocities, muscle and tendon strain, blood flow velocities, artery wall velocities and displacement, artery wall blood pressure pulse wave velocity, lung motion, cardiac kinematics and blood flow, or brain tissue motion and blood flow.

18. The apparatus of claim 11, wherein the processor-executable instructions, when executed by the one or more processors, further cause the apparatus to:
determine, based on the range information and the kinematic information, one or more medical conditions associated with the individual, wherein the one or more medical conditions comprise one or more of one or more cardiovascular conditions, peripheral vascular conditions, cerebrovascular conditions, or musculoskeletal conditions;
output one or more notifications associated with the one or more medical conditions; and
cause, based on the one or more medical conditions, one or more treatments associated with the individual, wherein the one or more treatments comprise one or more of administering medication or administering a medical procedure.

19. The apparatus of claim 11, wherein the processor-executable instructions, when executed by the one or more processors, further cause the apparatus to:
outputting, based on the range information and the kinematic information, one or more signals for controlling an external device, wherein the external device comprises a powered prosthetic limb, an exoskeleton, or one or more human machine interfaces, wherein the one or more human machine interfaces comprise one or more of feedback for neurostimulation, neuromuscular electrical stimulation, or functional electrical stimulation.

20. The apparatus of claim 11, wherein the processor-executable instructions, when executed by the one or more processors, further cause the apparatus to:
generate, based on the range information and the kinematic information, image data associated with the one or more locations on the individual, wherein the image data comprises one or more M-mode ultrasound images.
